(19) United States
(12) Patent Application Publication
(10) Pub. No.: US 2020/0165617 A1
(43) Pub. Date: May 28, 2020

(54) MICRORNAS FOR TREATMENT OF NEUROPATHIC PAIN

(71) Applicant: New York University, New York, NY (US)

(72) Inventor: Esperanza RECIO-PINTO, Bayside, NY (US)

(21) Appl. No.: 16/612,307

(22) PCT Filed: May 9, 2018
(86) PCT No.: PCT/US2018/031877

§ 371 Date: Nov. 8, 2019

Related U.S. Application Data
(60) Provisional application No. 62/503,688, filed on May 9, 2017.

(51) Int. Cl. C12N 15/113; A61P 25/02

(52) U.S. Cl. CPC C12N 15/1138; C12N 2820/60; A61P 25/02

(57) ABSTRACT

Provided are methods for treatment of neuropathic pain. The method comprises providing to an individual in need of treatment an effective amount of miR-133b-3p miRNA and/or miR-143-3p miRNA, with or without miR-1a-3p miR-NA. Compositions comprising these miRNAs, their precursors of vectors encoding the miRNAs are also provided.

### CROSS REFERENCE TO RELATED APPLICATIONS

This application claims priority to U.S. Provisional patent application No. 62/503,688, filed on May 9, 2017, the disclosure of which is incorporated herein by reference.

### BACKGROUND OF THE INVENTION

Despite concerted efforts to study, treat, and prevent chronic neuropathic pain, treatment of this type of pain remains relatively unsuccessful in about 2-10% of surgical patients. Chronic post-surgical pain is associated with peripheral nerve injury. Peripheral nerve injury is thought to result in expression changes of many proteins. Thus effective treatment for peripheral nerve injury-induced refractory neuropathic pain may require the simultaneous modification of many proteins. An approach that has recently gained attention as a way to target multiple proteins is via the modulation of microRNA (miRNA) expression. As with protein changes, miRNA changes following nerve injury may not be specific to chronic neuropathic pain. Different approaches have been taken to select potentially neuropathic pain-relevant miRNAs. Some groups have selected miRNAs that target molecules involved in inflammatory processes at the spinal cord level. This approach has revealed that the modulation of some of those miRs (miR-195, miR-146a-5p, miR-221, miR-155, miR-19a, miR-124) by using intrathecal injections can decrease neuropathic pain following lumbar-spinal nerve ligation or sciatic nerve injury (Lu et al., Brain, Behavior, and Immunity, 2015, 49:119-129; Shi et al., Glia, 2013, 61(4):504-512; Tan et al., Neurochemical Research 2015, 40(3):550-560; Wang et al., International Journal of Clinical and Experimental Pathology 2015, 8(9):10901-10907; Willemen et al., Journal of Neuroinflammation 2012, 9:143; Xia et al., J Mol Neurosci 2016). Another approach has been to select miRNAs that target ion channels. Daily intrathecal injections of miR-103, a miRNA that targets L-type calcium channels in the spinal cord, decreased neuropathic pain following spinal nerve ligation (Favereaux et al., The EMBO journal 2011, 30(18):3830-3841). Single intrathecal injections of miR-183 (Lin et al., The European Journal of Neuroscience 2014, 39(10):1682-1689) or miR-96 (Chen et al., Neurochemical Research 2014; 39(1):76-83), and injection into the dorsal root ganglion (DRG) of miR7a (Sakai et al., Brain 2013, 136(Pt 9):2738-2750), miRNAs that target sodium channels at the DRG, were shown to decrease neuropathic pain in the spinal nerve ligation or the chronic constriction sciatic nerve models. These approaches showed efficacy in the paradigms evaluated for hours or up to few days, but sustained long-term relief was not achieved.

### SUMMARY OF THE DISCLOSURE

The present disclosure is directed to methods of treatment of pain, including chronic pain. For example, the present methods and compositions can be used to treat neuropathic pain. The method comprises administering to an individual in need of treatment an effective amount of one or more polynucleotides comprising miR-133b-3p miRNA and/or miR-143-3p miRNA, or precursors thereof, with or without miR-1a-3p miRNA or precursors thereof. The polynucleotides may be DNA or expression vectors encoding the miRNAs. The precursors result in the production of both miR-133-3p and miR-133-5p; or miR-143-3p and miR-143-5p, or miR-1a-3p and miR-1a-5p.

As described in an example herein, administration of the precursors of miR-133b-3p or miR-143-3p within a short time after the injury (such as up to 3 days) prevented the development of persistent mechanical and cold allodynia. Administration of individual miR precursors (of miR-133b-3p or miR-143-3p) later (such as at or after 3 days) resulted in relief of pain, but the relief was not sustained. However, if the precursor of miR-1a-3p was also administered in addition to the precursor of miR-133b-3p and/or miR-143-3p, beyond the 3 day period, again, sustained relief of pain could be achieved.

In an aspect, this disclosure provides a method of treating neuropathic pain comprising administering to an individual in need of treatment an effective amount of one or more of the following polynucleotides: i) miR-133b-3p miRNA or precursor thereof, or a DNA polynucleotide encoding the miRNA or the precursor thereof; ii) miR-143-3p miRNA or precursor thereof, or a DNA polynucleotide encoding the miRNA or the precursor thereof; iii) an expression vector encoding the miR-133b-3p miRNA, or a precursor thereof, and iv) an expression vector encoding the miR-143-3p miRNA, or precursor thereof. When administered shortly after an injury (about 72 hours or less from the time of injury), the miR-133b-3p or the miR-143-3p miRNA alone can provide sustained relief from neuropathic pain. In one embodiment, both miR-133b-3p and the miR-143-3p miRNA may be administered. The method may further comprise administering to the individual miR-1a-3p miRNA, a precursor thereof, or a DNA polynucleotide encoding the miR-1a-3p miRNA or the precursor thereof, or an expression vector encoding miR-1a-3p miRNA or a precursor thereof. The addition of miR-1a-3p miRNA is particularly useful when the miR-133b-3p miRNA and/or the miR-143-3p miRNA are administered after more than 72 hours following an injury. The various miRs or precursors thereof, or polynucleotides encoding them can be administered together or separately, at the same time or different times.

In one aspect, this disclosure provides an expression vector encoding a miRNA or a precursor thereof, wherein the miRNA is miR-133b-3p, miR-143-3p, or miR-1a-3p. The expression vector may be a lentiviral vector or herpes simplex virus (HSV) vector, which may encode the miRNA or a precursor thereof. The expression vectors may be provided in a pharmaceutical composition. In embodiments, introduction of an expression vector facilitates expression of the miRNA in cells that received the expression vector. In embodiments, a sequence encoding an miRNA is integrated into a chromosome of a cell.

The compositions and method of this disclosure may be used for providing temporary or sustained relief of neuropathic pain, such as pain resulting from peripheral nerve injury.

### BRIEF DESCRIPTION OF THE FIGURES

FIG. 1. Rats that do not develop chronic pain following a peripheral nerve injury display up-regulation of miR-133b-3p, miR-143-3p and miR-1a-3p. Levels of miR-143-3p, miR-133b-3p, and miR-1a-3p following Sural-SNI or Tibial-SNI in the ipsilateral (IL) (A,B,C) and contralateral (CL) (D,E,F) DRG. Fold change=(2^{-ΔΔCT}). ΔΔCT=experimental ΔC_{T} (Sural-SNI or Tibial-SNI) minus sham ΔC_{T}. Reference miRNAs: snoRNA135-4380912 and U87-4386735. Black symbols: Sural-SNI vs Tibial-SNI at a given day, Two-way-Anova, Fisher's post-test; Gray symbols: SNI vs “0” (the “Sham” value), One sample t-test; *p<0.001, # p<0.01, @p<0.02, +p<0.05. Data mean±sem (for this and all figures).

FIG. 2. A single i.t. injection of LV-miR-143-3p or LV-miR-133b-3p, but not of LV-miR-1a-3p, on the day of the surgery prevents the development of mechanical and cold allodynia. (A) Protocol: i.t. injection done 10-15 min following surgery (dotted line). Symbol key of injected transduction units (TU) for (B-E). (B) Mechanical and (C) cold allodynia in the IL paws. (D) Mechanical and (E) cold allodynia in the CL paws. Sural-SNI-LV-miR-143-3p and Sural-SNI-LV-miR-133b-3p n=5, Sural-SNI-LV-miRla-3p n=4, Sural-SNI-LV-scramble-miRNA n=8 up to day 6, then n=5 up to day 48, n=2 for days 83-112. Group vs Sural-SNI-scramble-miRNA \hat{ }p<0.0001,*p<0.001, # p<0.01, Two-way Anova, Dunnett's post-test.

FIG. 3. A single i.t. injection of LV-miR-143 or LV-miR-133 at day 3 post-surgery has different effects on mechanical and cold allodynia. (A) Protocol: injection at day 3 post-surgery. Naïve-scramble-miR (Naïve). (B) Mechanical and (C) cold allodynia in IL paws, Sural-SNI-LV-miR-143-3p. Two-way-Anova, Dunnett's post-test, Sural-SNI-LV-miR-143-3p vs the Sural-SNI-scramble-miR, \hat{ }p<0.0001, *p<0.001, # p<0.01, @p<0.02, +p<0.05. For cold allodynia, Sural-SNI-10⁵ or 10⁶ TU were no different vs Naïve. Different but not indicated: (i) mechanical allodynia: Sural-SNI groups vs Naïve; (ii) cold allodynia: Sural-SNI-scramble-miRNA vs Naïve; Sural-SNI-10³ TU vs Naïve at days 28, 35 and 48. (D) Mechanical and (E) cold allodynia in the CL paw for Sural-SNI-LV-miR-143-3p. (F) Mechanical and (G) cold allodynia in the IL paw for Sural-SNI-LV-miR-133b-3p. Two-way-Anova, Dunnett's post-test, group vs the Sural-SNI-scramble-miRNA, \hat{ }p<0.0001, *p<0.001, # p<0.01, @p<0.02, +p<0.05. For cold allodynia Sural-SNI-10⁵ TU was not different vs Naïve. Different but not indicated: (i) mechanical allodynia: Sural-SNI groups vs Naïve; (ii) cold allodynia: Sural-SNI-scramble miR vs Naïve; Sural-SNI-10³ TU vs Naïve. (H) Mechanical and (I) cold allodynia in the CL paw for Sural-SNI-LV-miR-133b-3p. (B-I) Sural-SNI-scramble-miRNA n=8 up today 6, then n=5. LV-miR-133b-3p at 1×10³ n=4, other groups n=3.

FIG. 4. A single i.t. injection of LV-miR-1 at day 3 post-surgery has no effects on mechanical or cold allodynia. Injections were done as described on FIG. 3A. (A) Mechanical and (B) cold allodynia in IL paws. (C) Mechanical and (D) cold allodynia in CL paws.

FIG. 5. A single i.t. injection of LV-miR-133 with miR-1 (miR-1333+miR-1), and of miR-143 with miR-1 (miR-143+miR-1), but not of miR-133 with miR-143 (miR-133+miR-143), at day 3 post-surgery produced a sustained decrease in mechanical allodynia. Injections were done as shown in (A). (B) Mechanical allodynia IL paw, (C) cold allodynia in IL paw (D) Mechanical allodynia CL paw, and (E) cold allodynia in CL paw These three miR combinations produced a sustained decrease in cold allodynia.

FIG. 6. Schematic representation of the observations. Further details are provided under the Detailed Description section.

FIG. 7. LV-miR-133 and LV-miR-143 do not lead to recovery of the injury-induced decrease in toe-spread. (A) Toe-spread before (BS) and post- (PS) surgery in the IL paw when i.t. injections were done on day 0. Naïve and Sural-SNI-scramble-miRNA, n=3; Sural-SNI-LV-miR-143-3p and Sural-SNI-LV-miR-133b-3p, n=5. For day 3 and day 48, naïve vs all sural-SNI (\hat{ }p<0.0001); group vs sural-SNI-scramble miRNA (+p<0.05). (B) Toe-spread BS and PS in the IL paw when i.t. injections were done of day 3 post-surgery. miR-133b-3p 1×10³ n=4, other groups n=3. Day 3 and Day 48, naïve vs Sural-SNI groups (\hat{ }p<0.0001). One-way-Anova, Tukey's post-test. In (A), the bars in each set for BS, Day 3 PS, and DAY 48 PS from left to right are Naïve IL scramble miR 1×10⁶, Sural-SNI IL scramble miR 1×10⁶, Sural-SNI IL miR-143 5×10⁵, Sural-SNI IL miR-133 2×10⁵. In (B), the bars in each set for BS, Day 3 PS, and DAY 48 PS from left to right are Naïve IL scramble miR 1×10⁶, Sural-SNI IL scramble miR 1×10⁶, Sural-SNI IL miR-143 1×10⁶, Sural-SNI IL miR-143 1×10⁵, Sural-SNI IL miR-143 1×10³, Sural-SNI IL miR-133 1×10⁵, Sural-SNI IL miR-133 1×10³.

FIG. 8. Expression of miRNAs following treatment with LV-miRs in DRG cell cultures and DRG isolated from sural SNI rats. One day old DRG cell cultures were transfected with either scramble-miRNA (A), LV-miR-133b-3p (B), or LV-miR-143-3p (C). Pictures were taken on live cells, 5 days post-transfection. Transfected cells are GFP positive. Scale bar 100 magnification 10×. Measurements of (D) mouse pri-miR-133b-3p, rat pri-miR-133b-3p, and mature miR-133b-3p; and (E) mouse pri-miR-143-3p in DRG cell cultures following 7 days post-transfection. “Fold change” (2^{-ΔΔCT}): ΔC_{T} from LV-miR transfected cells minus the ΔC_{T} from untransfected cells. Hence there should be no significant change for rat-pri-miR (between transfected and untransfected cells; “log 2 (fold change=0). One sample t-test vs hypothetical value=0, *p<0.001, @p<0.02 (n=3 cultures). (F) Measurements of mouse pri-miR-133b-3p and rat pri-miR-133b-3p in DRG isolated from sural-SNI at day 7 and at day 57 (sural-SNI) following the i.t. injection with LV-miR-133b-3p. The “fold change” (2^{-ΔΔCT}) was obtained by substracting the ΔC_{T} from LV-miR-133b-3p injected sural-SNI rats minus the ΔC_{T} from LV-scramble-miRNA injected sural SNI rats. Unpaired t-test one tailed $p=0.0573; # p=0.0144 (n=3 rats). (D-F) GAPDH was used as the endogenous reference to obtain the ΔC_{T} value for the pri-miRNAs; U87 and snoRNA135 were used as the endogenous reference to obtain the ΔC_{T} value for miR-133b-3p.

FIG. 9. In cultured DRG neurons increasing the levels of miR-133b-3p and miR-143-3p reduce neuronal excitability. (A) Under current clamp, current pulses of increasing magnitude (50 pA increments) were applied, and the changes in membrane potential recorded. (B) % cells with action potentials (AP); cell area, resting potential (Vr), threshold potential (Vth), lower magnitude of current injected (I injected) to generate an AP. One-way Anova, Tukey's multiple post-test: a: vs miR-143-3p, p=0.0372, vs control p=0.1146; b: vs miR-133b-3p p=0.1144, vs control p=0.0388. (C) Area distribution of the cells used. Number of DRG cell culture preparations: 8, 5, 6, 8, 5 for control, miR-133b-3p, anti-miR-133b-3p, miR-143-3p, and anti-miR-143-3p, respectively.

FIG. 10. In cultured DRG neurons miR-133b-3p and miR-143-3p, but not miR-1, increased the depolarization-evoked increase in [Ca²⁺]_{cyt}. (A) Traces of transient increases in [Ca²⁺]_{cyt} evoked by 10 sec pulses of KCl (50, 20 and 30 mM), interval between pulses was 10 minutes. (B-G) Areas of K-evoked [Ca²⁺]_{cyt} increase (n)=cell number. (B and E) Effects of miR-133b-3p and anti-miR-133b-3p. (C and F) Effects of miR-143-3p and anti-143-3p. (D and G) Effects of miR-1 and anti-miR-1. The D'Agostino-Pearson omnibus normality test shows that the data does not have a Gaussian distribution. One-way Anova using the noparametric Kruskal-Wallis testm abd the Dunn's post-test. \hat{ }p<0.0001, *p<0.001, # p<0.05. For each miR family (control, miR and anti-miR) 3 separate preparations were used; from each slide two separate fields were used. For the miR-133 group: control (8 slides, 16 fields, 111 cells); miR-133 (7 slides, 14 fields, 92 cells); anti-miR-133 (7 slides, 13 fields, 131 cells). For the miR-143 group: control (6 slides, 12 fields, 74 cells); miR-143 (6 slides, 11 fields, 81 cells); anti-miR-143 (6 slides, 13 fields, 86 cells). For the miR-1 group: control (3 slides, 6 fields, 43 cells); miR-1 (6 slides, 12 fields, 79 cells); anti-miR-1 (6 slides, 12 fields, 92 cells).

FIG. 11. MiR-133b-3p has a positive effect while miR-143-3p has a negative effect on neurite outgrowth (A) Freshly dissociated DRG cell cultures were transfected with either no miRNAs (control), miR-133b-3p or with miR-143-3p. Three days following transfection neurons were immunostained with anti-β-III-tubulin (red), scale bar 50 μm. Magnification 20×. (B) Number of neurites per cell (n=# cells); (C) neurite length (n=# neurites, from the same cells as in B). (B,C) One-way Anova, Sidak's post-test \hat{ }p<0.0001, *p<0.001, # p<0.01, @p<0.02.

FIG. 12. The actions of miR-133b-3p and miR-143-3p on 3′UTR-Scn2b and 3′UTR-Trpm8 are similar and differ from those of miR-1a-3p. MiR 3′UTR target clones containing a Gaussian luciferance (Gluc) reporter gene were used (A) Because of his length two clones were used for the 3′UTR-Scn2b (sodium voltage-gated channel beta subunit): 3′UTR-Scn2b(a) and 3′-UTR-Scn2b(b) (B) 3′UTR-TRPM8 (transient receptor potential cation channel subfamily M member 8). (C) 3′UTR-Piezo2 (piezo type mechanosensitive ion channel component 2). These clones contain a constitutively expressed secreted alkaline phosphatase (SEAP) reporter gene that is used as an internal control. n=number of transfections. For each transfection two wells were used for each condition. For each transfection, the Gluc/SeAp ratio of each well was measured, averaged, and normalized to the ratio value obtained with the “plasmid alone” group. To correct for non-3′UTR interactions, the normalized Gluc/SeAP values obtained with the 3′UTR-plasmid were corrected using the normalized Gluc/SeAP values obtained with the target control vector (without 3′UTR). One way Anova-Fisher's post-test vs negative-miR *P<0.005, # P<0.01, \hat{ }P<0.02, +P<0.05.

### DETAILED DESCRIPTION OF THE INVENTION

The present disclosure provides miRNA based treatments for chronic pain, such as pain caused by peripheral nerve injuries. In a method of the present disclosure, administration of miR-133b-3p or miR-143-3p, or precursors thereof, or vectors encoding the miRs early after injury prevents the development of mechanical and cold allodynia. The inhibitory effect is sustained over several months. If not administered early after injury, administration of miR-133b-3p or miR-143-3p still produced sustained reduction in cold allodynia. If additionally, miR-1a-3p miRNA was also administered with miR-133b-3p, or miR-143-3p, or both, at later times after injury, sustained reversal of both cold and mechanical allodynia can be achieved.

The disclosure includes all nucleotide sequences referred to herein, their complementary sequences, and DNA equivalents of RNA sequences. The sequences can include mutational deletions and insertions, so long as the sequences retain function to reduce chronic pain. Polynucleotides delivered to an individual can comprise or consist of the sequences disclosed herein. In embodiments, a contiguous segment of at least 10 nucleotides or any sequences disclosed herein is included in the disclosure. In embodiments, the miRNA sequences are 15 to 25 nucleotides.

As used herein, an “effective amount” means an amount of an agent sufficient to achieve, in a single or multiple doses, the intended purpose of treatment.

As used herein, “treatment” means reducing the severity of one or more of the symptoms associated with the indication that the treatment is being used for. Thus treatment includes ameliorating one or more symptoms associated with an indication. The term “treatment” also includes relapse, or prophylaxis as well as the treatment of acute or chronic signs, symptoms and/or malfunctions. The treatment can be orientated symptomatically, for example, to suppress symptoms. It can be effected over a short time period, over a medium time period, or it can be a long-term treatment, for example within the context of a maintenance therapy.

As used herein, “neuropathic pain” means pain resulting from pathological changes, functional changes, or injuries to the nervous system, such as the peripheral nervous system. Neuropathic pain may result from surgery, trauma, underlying diseased conditions including cancer (including chemotherapy induced neuronal damage), and diabetes.

The present disclosure provides a method of treating pain, such as neuropathic pain, including chronic neuropathic pain, comprising administering to an individual in need of treatment an effective amount of one or more of the following: miR-133b-3p miRNA (also referred to herein as miR-133); miR-143-3p miRNA (also referred to herein as miR-143), precursors of these miRNAs, or one or more expression vectors encoding miR-133 or miR-143 or precursors thereof. The method may further comprise administering to an individual an effective amount of miR-1a-3p miRNA (also referred to herein as miR-1), or a precursor of miR-1, or an expression vector encoding miR-1 or a precursor thereof. In one embodiment, a mixture of miRNA comprising miR-133 and miR-143 may be administered. In one embodiment, a mixture of miRNA comprising miR-133 and miR-1 may be administered. In one embodiment, a mixture of miRNA comprising miR-143 and miR-1 may be administered. In one embodiment, a mixture of miRNA comprising miR-133, miR-143 and miR-1 may be administered. During a treatment regimen, different combinations of the miRs may be administered at different times. The combinations may be administered as a single injection or may be administered as separate injections.

The sequences of miR-133b miRNA, miR-143 miRNA and miR-1a miRNA are well-known in the art. For example, they are available at mirbase.org. The sequences and all variants thereof are incorporated herein by reference as of the filing date of this application. The mature sequences for mouse (mmu); rat (rno) and human (hsa) are:

miR-133b-3p:
mmu/rno/hsa:
(SEQ ID NO: 1)
UUUGGUCCCCUUCAACCAGCUA
miR-133b-5p:
mmu/rno/hsa:
(SEQ ID NO: 2)
GCUGGUCAAACGGAACCAAGUC
miR-143b-3p:
rno:
(SEQ ID NO: 3)
UGAGAUGAAGCACUGUAGCUCA
mmu/hsa:
(SEQ ID NO: 4)
UGAGAUGAAGCACUGUAGCUC
miR-143b-5p:
rno/mmu:
(SEQ ID NO: 5)
GGUGCAGUGCUGCAUCUCUGG
hsa:
(SEQ ID NO: 6)
GGUGCAGUGCUGCAUCUCUGGU
miR-1a-3p:
rno:
(SEQ ID NO: 7)
UGGAAUGUAAAGAAGUGUGUAU
mmu/hsa:
(SEQ ID NO: 8)
UGGAAUGUAAAGAAGUAUGUAU
miR-1a-5p:
rno:
(SEQ ID NO: 9)
GCACAUACUUCUUUAUGUACCC
mmu(1):
(SEQ ID NO: 10)
ACAUACUUCUUUAUAUGCCCAUA
mmu(2):
(SEQ ID NO: 11)
ACAUACUUCUUUAUAUACCCAUA
hsa:
(SEQ ID NO: 12)
GUACAUACUUCUUUAUGUACCCAUA

The pre-miRNA sequences for mouse (mmu); rat (rno) and human (hsa) are.

Pre-mmu-mir-133b
(SEQ ID NO: 13)
CCUCCAAAGGGAGUGGCCCCCUGCUCUGGCUGGUCAAACGGAACCAAG
UCCGUCUUCCUGAGAGGUUUGGUCCCCUUCAACCAGCUACAGCAGGGC
UGGCAAAGCUCAAUAUUUGGAGA
Pre-rno-mir-133b
(SEQ ID NO: 14)
CCCUGCUCUGGCUGGUCAAACGGAACCAAGUCCGUCUUCCUGAGAGGU
UUGGUCCCCUUCAACCAGCUACAGCAGGGCUGGCAA
Pre-hsa-mir-133b
(SEQ ID NO: 15)
CCUCAGAAGAAAGAUGCCCCCUGCUCUGGCUGGUCAAACGGAACCAAG
UCCGUCUUCCUGAGAGGUUUGGUCCCCUUCAACCAGCUACAGCAGGGC
UGGCAAUGCCCAGUCCUUGGAGA
Pre-rno-mir-143
(SEQ ID NO: 16)
GCGGAGCGCCUGUCUCCCAGCCUGAGGUGCAGUGCUGCAUCUCUGGUC
AGUUGGGAGUCUGAGAUGAAGCACUGUAGCUCAGGAAGGGAGAAGAUG
UUCUGCAGC
Pre-mmu-mir-143
(SEQ ID NO: 17)
CCUGAGGUGCAGUGCUGCAUCUCUGGUCAGUUGGGAGUCUGAGAUGAA
GCACUGUAGCUCAGG
Pre-hsa-mir-143
(SEQ ID NO: 18)
GCGCAGCGCCCUGUCUCCCAGCCUGAGGUGCAGUGCUGCAUCUCUGGU
CAGUUGGGAGUCUGAGAUGAAGCACUGUAGCUCAGGAAGAGAGAAGUU
GUUCUGCAGC
Pre-rno-mir-1
(SEQ ID NO: 19)
UGCCUACUCAGAGCACAUACUUCUUUAUGUACCCAUAUGAACAUAGAA
UGCUAUGGAAUGUAAAGAAGUGUGUAUUUUGGGUAGGUA
Pre-mmu-mir-1a-2
(SEQ ID NO: 20)
UCAGAGCACAUACUUCUUUAUGUACCCAUAUGAACAUUCAGUGCUAUG
GAAUGUAAAGAAGUAUGUAUUUUG
Pre-hsa-mir-1-1
(SEQ ID NO: 21)
UGGGAAACAUACUUCUUUAUAUGCCCAUAUGGACCUGCUAAGCUAUGG
AAUGUAAAGAAGUAUGUAUCUCA
Pre-hsa-mir-1-2
(SEQ ID NO: 22)
ACCUACUCAGAGUACAUACUUCUUUAUGUACCCAUAUGAACAUACAAU
GCUAUGGAAUGUAAAGAAGUAUGUAUUUUUGGUAGGC

RNA polynucleotides delivered to an individual (such as a human) according to this disclosure can in various stages comprise unprocessed or processed RNA transcripts that are transcribed from the pertinent miRNA-coding gene. An unprocessed miRNA transcript is an example of a miRNA precursor of this disclosure, and is sometimes referred to as an miRNA precursor, one example of which is a pri-miRNA. A pri-miRNA may be a single stranded RNA polynucleotide that adopts RNA stem loop secondary structures and can comprise one or more miRNA segments. Such pri-miRNA precursors generally comprise approximately 60 to 120 RNA nucleotides. For example, the precursor may have 70-100 nucleotides. It is recognized in the art that such pri-miRNA precursors can be converted into pre-miRNA hairpins, which are subsequently processed by an RNAse into an active 19-25 nucleotide miRNA molecule. This 19-25 nucleotide RNA molecule is sometimes referred to as a mature miRNA. The disclosure accordingly comprises in certain embodiments delivering miRNA precursors, including pri-mRNA, and pre-miRNA directly to an individual, or expression vectors which encode miRNA precursors or mature miRNAs. Each of these approaches is considered to comprise a method of delivering an miRNA to an individual, i.e., whether an miRNA or precursor thereof is delivered directly or by an expression vector that encodes the precursor or the miRNA, the mature miRNA will ultimately be present in a cell and will function to downregulate its target(s) to produce the prophylactic and/or therapeutic effects on pain as described herein.

The expression vectors can be comprised of DNA or RNA. The expression vectors can be viral vectors, including but not limited to retroviral vectors, e.g., lentiviral vectors in the case of RNA viral vectors, or herpes simplex virus (HSV) vectors in the case of DNA viral vectors, or polymer-coated viral vectors in the case of hybrid vectors. Commercially available viral vectors may be adapted for making and using polynucleotides disclosed herein for an individual in need thereof. Suitable expression vectors can contain a variety of control sequences, which refer to nucleic acid sequences necessary or useful for the transcription of an operably linked coding sequence in a particular host cell. Such sequences may include promotors, enhancers, initiation signals, multiple cloning sites, splicing sites, termination signals, origins of replication, selectable markers, and the like. The vectors may facilitate delivery to primary sensory neurons, and the retrograde transport in primary sensory.

The expression vectors may be capable of expressing the nucleic acid molecules either permanently or transiently in target cells. The expression vectors thus may be such that they are maintained within cells episomally, or a segment of the construct encoding at least the miRNA sequence may be integrated into the genome of the recipient cells, thereby facilitating sustained expression.

The miRNAs or vectors encoding them or encoding their precursors can be generated by routine methods. In the case of viral delivery vectors, viruses can be grown in any suitable cell culture, using helper viruses in such cases where helper viruses are required, and separated from the cell cultures. The nucleic acids and/or intact viruses may be isolated or purified prior to their being used for administration and introduction into a cell. Viruses can be titered to achieve any desirable number, amount, infectious units, etc. for use in methods of this disclosure.

Introduction into a cell for viral production and for the prophylactic and/or therapeutic approaches of this disclosure can be carried out by known methods, such as, for example, transfection, transduction, infection and other methods known for introducing nucleic acids such as expression vectors, including viral expression vectors, into a cell and into cell populations.

The disclosure provides compositions comprising an effective amount of one or more miRNAs or vectors encoding them or their precursors disclosed herein in pharmaceutically acceptable carriers. Viral particles comprising recombinant viral genomes which encode the miRNA or an miRNA precursor can be administered, and/or nucleic acids can be directly administered.

Pharmaceutically acceptable carriers may include a diluent, adjuvant, excipient, or other vehicle with which the therapeutic is administered. Some examples of materials which can serve as pharmaceutically-acceptable carriers include: sugars, such as lactose, glucose and sucrose; starches, such as corn starch and potato starch; cellulose, including sodium carboxymethyl cellulose, ethyl cellulose and cellulose acetate; powdered tragacanth; malt; gelatin; talc; excipients, such as cocoa butter and waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The composition, if desired, can also contain minor amounts of wetting or emulsifying agents, or pH buffering agents. Some examples of compositions suitable for mixing with the agent can be found in: Remington: The Science and Practice of Pharmacy (2005) 21st Edition, Philadelphia, Pa. Lippincott Williams & Wilkins. In one embodiment, the agent is substantially purified (e.g., substantially free from substances that limit its effect or produce undesired side-effects).

Compositions may be formulated for intrathecal, parenteral, intravenous, or intramuscular delivery, or topical or transdermal use. Intrathecal delivery is a particularly useful way for delivery of compositions provided herein. By this method, the compositions are released into the surrounding CSF and/or tissues and can penetrate into the spinal cord parenchyma. Intrathecal delivery of nucleic acid compositions can help to keep expression local. The present compositions can also be delivered to dorsal root ganglia (DRG) neurons by injection into the epidural space. The nucleic acid compositions may be delivered via intrathecal cannulation. The location of the intrathecal delivery can be that corresponding to the location of the DRG containing the somas of the injured peripheral nerves. For example, the level location of the delivery of the injection may be selected depending upon which nerves are injured, e.g., thoracic, lumbar, cervical etc.

Dosage forms for the topical, transdermal administration include powders, sprays, ointments, pastes, creams, lotions, gels, solutions, and patches. The compositions may be aqueous compositions. Saline solutions and aqueous dextrose and glycerol solutions may be employed as liquid carriers.

Delivery aids may be used for delivery of the compositions. Such aids may comprise components that facilitate release of the agents over certain time periods and/or intervals, and can include compositions that enhance delivery of the agents. For example, nanoparticle, microsphere or liposome formulations, a variety of which are known in the art and are commercially available, may be used. In illustrative embodiments, an expression vector as described herein is present in a delivery vehicle, where the delivery vehicle is selected from a polymeric carrier, a micelle, a liposome, a lipoplex, a polyplex, a peptide, a polymer, a dendrimer, lipids, or a nanoparticle. For example, lipofectamine is used.

The amount of polynucleotides for treatment can be determined by a clinician or other health care provider. As an example, 0.1 to 1×10′ transduction units can be used when using viral vectors. When using mature form of miRNA or the precursor without a viral vector, then 0.01 to 10 μg can be used.

The present compositions can be delivered at desired times. In one embodiment, the compositions are delivered as soon as feasible after the injury. For example, the compositions may be delivered within a few hours, such as within 3, 6, 12, 18 or 24 hours, or 1, 2 or 3 days of the injury, or the compositions may be delivered at a later time, such as any time when neuropathic pain develops. For example, compositions may be delivered after 3 months, 6 months, 12 months, or more after an injury.

The present compositions may be used for the treatment of neuropathic pain, including chronic neuropathic pain, allodynia, including cold and mechanical allodynia, heat hyperalgesia, spontaneous pain and the like. While the present methods are described for use in humans, they may be used for non-human animals also.

The present compositions can provide short-term and/or long term treatment of neuropathic pain. The pain may be short term or long term. If administered within a short time after an injury (such as injury to peripheral nerve), then the 143 miRNA, the 133 miRNA or both can be used and can provide sustained relief from nerve pain. If administered beyond a 3 day period after injury, then the 143 miRNA and the 133 miRNA can still provide short term relief for some neuropathic pain, but for sustained long term, it is preferable to also administer miR-1 miRNA together with the miR-143 miRNA or the miR-133 miRNA, or both. While a single injection of the miRNAs (of each one if multiple types of miRNA are injected) may be sufficient, over time if needed, additional administrations can also be carried out.

Some specific embodiments are provided below.

In an embodiment, the present disclosure provides sustained relief from chronic neuropathic pain, such as pain caused by peripheral nerve injury comprising administering to an individual in need of treatment an effective amount of miR-143-3p miRNA or precursor thereof, or a DNA polynucleotide encoding the miRNA or the precursor thereof, shortly after a peripheral nerve injury, such as, for example within 72 hours of the injury. For example, the miRNAs or precursors thereof, or DNA polynucleotides encoding same may be administered immediately (within minutes) after the injury or within 1, 2, 3, 6, 9, 12, 18, 24, 30, 36, 42, 48, 60, or 72 hours of the injury. In an embodiment, the miR-143-3p miRNA or precursor thereof, or a DNA polynucleotide encoding the miRNA or the precursor thereof may be the only miRNA or polynucleotide administered.

In an embodiment, the present disclosure provides sustained relief from chronic neuropathic pain, such as pain caused by peripheral nerve injury comprising administering to an individual in need of treatment an effective amount of miR-133b-3p miRNA or precursor thereof, or a DNA polynucleotide encoding the miRNA or the precursor thereof, within a short time after a peripheral nerve injury, such as, for example within 72 hours of the injury. For example, the miRNAs or precursors thereof, or DNA polynucleotides encoding same may be administered immediately (within minutes) after the injury or within 1, 2, 3, 6, 9, 12, 18, 24, 30, 36, 42, 48, 60, or 72 hours of the injury. In an embodiment, the miR-133b-3p miRNA or precursor thereof, or a DNA polynucleotide encoding the miRNA or the precursor thereof may be the only miRNA or polynucleotide administered.

In an embodiment, the present disclosure provides sustained relief from chronic neuropathic pain, such as pain caused by peripheral nerve injury comprising administering to an individual in need of treatment an effective amount of miR-143-3p miRNA and miR-133b-3p miRNA or precursors thereof, or DNA polynucleotides encoding the miRNA or the precursors thereof, within a short time after a peripheral nerve injury, such as, for example within 72 hours of the injury. For example, the miRNAs or precursors thereor of DNA polynucleotides encoding same may be administered immediately (within minutes) after the injury or within 1, 2, 3, 6, 9, 12, 18, 24, 30, 36, 42, 48, 60, or 72 hours of the injury. In an embodiment, the miR-143-3p miRNA and miR-133b-3p miRNA or precursors thereof, or DNA polynucleotides encoding the miRNA or the precursors thereof may be the only miRNAs or polynucleotides administered.

In an embodiment, the present disclosure provides sustained relief from chronic neuropathic pain, such as pain caused by peripheral nerve injury comprising administering to an individual in need of treatment an effective amount of miR-143-3p miRNA and miR-1a-3p miRNA, or precursors thereof, or DNA polynucleotides encoding the miRNA or the precursors thereof, after about 3 days after a peripheral nerve injury, such as, for example after 72 hours of the injury. For example, the miRNAs or precursors thereof, or DNA polynucleotides encoding same may be administered within 7 days, such as after 3, 4, 5 or 6 days, or they may be administered after 7 days such as after 10 days, 12 days, 15 days, one month, 3 months or 6 months from the time of the injury. In an embodiment, the miR-143-3p miRNA and miR-1a-3p miRNA or precursors thereof, or DNA polynucleotides encoding the miRNA or the precursors thereof may be the only miRNAs or polynucleotides administered.

In an embodiment, the present disclosure provides sustained relief from chronic neuropathic pain, such as pain caused by peripheral nerve injury comprising administering to an individual in need of treatment an effective amount of miR-133b-3p miRNA and miR-1a-3p miRNA, or precursors thereof, or DNA polynucleotides encoding the miRNA or the precursors thereof, after about 3 days after a peripheral nerve injury, such as, for example after 72 hours of the injury. For example, the miRNAs or precursors thereof, or DNA polynucleotides encoding same may be administered within 7 days, such as after 3, 4, 5 or 6 days, or they may be administered after 7 days such as after 10 days, 12 days, 15 days, one month, 3 months or 6 months from the time of the injury. In an embodiment, the miR-133b-3p miRNA and miR-1a-3p miRNA or precursors thereof, or DNA polynucleotides encoding the miRNA or the precursors thereof may be the only miRNAs or polynucleotides administered.

In an embodiment, the present disclosure provides sustained relief from chronic neuropathic pain, such as pain caused by peripheral nerve injury comprising administering to an individual in need of treatment an effective amount of miR-143-3p miRNA, miR-133b-3p miRNA and miR-1a-3p miRNA, or precursors thereof, or DNA polynucleotides encoding the miRNA or the precursors thereof, after about 3 days after a peripheral nerve injury, such as, for example after 72 hours of the injury. For example, the miRNAs or precursors thereof, or DNA polynucleotides encoding same may be administered within 7 days, such as after 3, 4, 5 or 6 days, or they may be administered after 7 days such as after 10 days, 12 days, 15 days, one month, 3 months or 6 months from the time of the injury. In an embodiment, the miR-143-3p miRNA, miR-133b-3p miRNA and miR-1a-3p miRNA or precursors thereof, or DNA polynucleotides encoding the miRNAs or the precursors thereof may be the only polynucleotides administered.

In one embodiment, this disclosure provides an expression vector encoding an miRNA or a precursor thereof, wherein the miRNA is miR-133b-3p, miR-143-3p, or miR-1a-3p. The expression vector can be a lentiviral vector or herpes simplex virus (HSV) vector. The expression vectors may be provided in a pharmaceutical composition(s). The miRNAs may also be provided in pharmaceutical compositions.

The following examples are provided as illustrative of the present methods. These examples are not intended to be restrictive in any way.

### Example 1

This example demonstrates, using lentiviral (LV) vectors as an example, that intrathecal injection of a vector encoding miR-133b-3p (LV-miR-133b-3p) or miR-143-3p (LV-miR-143-3p) on the day of the injury prevents the development of strong sustained mechanical and cold allodynia (observation period over 100 days) in animals that normally develop chronic pain. In contrast, a similar intrathecal injection of either LV-miR-133b-3p or LV-miR-143-3p on day 3 post-injury produced a transient 50% reduction in mechanical allodynia, while it still resulted in a maintained >90% reduction in cold allodynia. In DRG neuronal cultures, these miRs reduced neuronal excitability, enhanced the depolarization-evoked cytoplasmic calcium increase, but had opposite effects on neurite outgrowth. The results indicate that early treatment with either miR-133b-3p or miR-143-3p can be used to evoke a sustained prevention of development of mechanical and cold allodynia.

Material and Methods

Plasmid Purification

The plasmids used to produce the lentivirus (LV) were purchased from SBI

(System Biosciences): mouse precursor scramble negative control construct (cat # MMIR-000-PA-1), mouse pre-microRNA expression construct miR-133b (cat # MMIR-133B-PA-1) and mouse pre-microRNA expression construct miR-143 (cat # MMIR-143-PA-1). For the purification of transfection-grade plasmids QlAfilter Plasmid Maxi kit (Qiagen, cat #12262) was used. All plasmids express the fluorescence protein GFP which was used to check the transfection efficiency of the lentiviral vectors.

Lentivirus and Animals

All the steps involved in the lentiviral (LV) vector production and usage were approved by the Institutional Biosafety Committee of New York University (NYU) Langone Medical Center and followed BSL2 containment protocols. Adult male Sprague-Dawley rats (250-400 g) were used following the guidelines approved by the NYU Langone Medical Center Institutional Animal Care and Use Committee (IACUC).

Cell Lines

The 293TN Human Kidney Producer Cell Line (SBI, cat # LV900A-1) was used for packaging and tittering.

Lentivector Packaging

LV-miR vectors (LV-miR00, LV-miR-133b, LV-miR-143, from systembio.com) were produced by con-transfecting 293TN cells with the helper plasmids pPACKH1 HIV (Lentivector Packaging kit, SBI cat # LV500A) and one of the specific miR-plasmid constructs (MMIR-000-PA-1, MMIR-133B-PA-1, MMIR-143-PA-1, or MMIR-1a-PA-1) by using the PureFection reagent (SBI, cat # LV750A) and following the manufacturer's instructions. The LV-vector containing supernatant was first centrifuged at 3000×g for 15 minutes to eliminate cell debris. Then it was concentrated 100 times by adding 1 volume of cold PEG-it Virus Precipitation Solution (SBI, cat # LV810A) (4° C.) to every 4 volumes of LV-vector-containing supernatant and spun at 1500×g for 30 minutes at 4° C. LV-vector pellets were re-suspended in cold, sterile Phosphate Buffered Saline (PBS) and stored at −80° C.

Titer of the LV-miR Preparations

293TN cells were plated at 1×10⁵ cells/well in a 24 wells plate, using complete DMEM medium (ThermoScientific, cat #11965-92) supplemented with 10% Fetal Calf Serum (cat #160000-044, Gibco), penicillin (100 U/ml) plus streptomycin (100 μg/ml) (Gibco, cat #15140-22), and 0.5 nM Glutamax (Gibco, cat #35050061)). After 24 hours, the medium of each well was replaced with 220 μl DMEM medium (without supplements) containing serial dilutions (from 10⁻² to 10^{-v}) of the LV-miR vector and incubated for 90 min at 37° C. Then, LV-miR was removed and 250 μl of complete DMEM medium was added to each well. After 72 hours the number of GFP positive cells were counted using a fluorescence microscope Zeiss Observer Z1 (Carl Zeiss, Germany) and the titer was calculated as Transducing Units per ml (TU/ml). In the figures, transduction units (TU) injected in a volume of 10 ul are indicated.

LV-miR Vector Transfection of Primary DRG Neuronal Cultures

DRG primary neuronal cultures were generated using lumbar DRG from adult male Sprague-Dawley rats as. Dissociated DRG cells were plated directly on 24 well plates by using Neurobasal medium (NBA) supplemented with 2% B27, penicillin (100 U/ml), streptomycin (100 μg/ml), and 0.5 mM Glutamax. After 24 hours, the medium of each well was replaced with 220 μl NBA medium (without supplements) containing either 10¹ or 10⁻² dilutions of the LV-miR vector preparation and incubated for 90 min at 37° C. Then 250 μl of NBA (with 2% B27 and 10% FCS) as added to each well. The transfection efficiency of the DRG cells (GFP positive cells) was analyzed by fluorescence microscopy at 48 and 72 hours.

Animal Model

Under isoflurane anesthesia, the sural spared-nerve injury (sural-SNI) was performed as previously described (Decosterd et al., Pain 2000; 87(2):149-158; Norcini et al., Frontiers in neuroscience 2014; 8:266). Briefly, the common peroneal and the tibial nerve Branches of the Sciatic Nerve were Ligated, Transected Distally to the Ligature, and 2-3 mm of each distal nerve branch stump was removed.

Intrathecal (i.t.) Injections

Naïve and Sural-SNI rats were randomly assigned to be injected with the LV-vector containing either the scramble miR (LV-miR00), LV-miR-133b-3p, LV-miR-143-3p, or LV-miR1a-3p. Intrathecal (i.t.) injections were performed as previously described (Mestre et al., J Pharmacol Toxicol Methods 1994; 32(4):197-200). Rats were initially anesthetized with 4.5% isofluorane for 5 minutes. A small incision in the lumbar area of the animal's back was made. Then isoflurane was decreased to 2.5% for the remainder of the procedure in order to observe the ‘tail flick’ response, which is an indicator of correct needle placement in the subarachnoid space. The rat was positioned at a 90° angle, to maximize the intervertebral space. The landmark points used for needle placement were the pelvic bones located at the level of the fifth lumbar vertebra. A 25 μl glass Hamilton syringe (cat #7636-01 with needle cat #7804-03 26 G RN 1″ 12⁰) was used to inject 8-16 μl of LV-miR vector solution prepared in sterile Phosphate Buffered Saline at concentrations ranging from 10⁵ to 10⁸ TU/ml. In the figures, the TU in a volume of 10 ul (volume injected) is indicated. The needle was inserted perpendicular to the vertebral column into the interlaminar space between the spinous processes of the L4 and L5 lumbar vertebrae. Following the injection, the syringe was held in position for 5-10 seconds and then slowly removed, to avoid leakage of the injected solution. The rat was placed back in the horizontal position and the skin was sutured.

Behavioral Test

Rats were marked on the top of their tails and were randomly placed into individual Plexiglas boxes located on an elevated metal grid allowing access to the plantar surface of the hind-paws. The investigator doing the measurements could not see the mark on the rat's tail. Mechanical thresholds (in grams) were measured in the sural region of the hind paws using the electronic von Frey apparatus equipped with a size 15 filament fitted on the 800 gram arm (IITC Life Sciences, Inc.). The individual applying the filament was different from the individual doing the read out on the electronic von Frey apparatus. Cold allodynia was evaluated by placing 20 μl of absolute acetone in the plantar surface of the hindpaws and measuring the duration of paw withdrawal. For each day, mechanical and cold allodynia measurements were repeated three times with an interval of about 5 min between stimuli, and for each animal the mean value was used.

Total RNA Extraction

Total RNA extraction from individual DRGs and cell DRG cultures was carried out as previously described (Norcini et al., Front Mol Neurosci 2016; 9:100; Norcini et al., Frontiers in neuroscience 2014; 8:266).

Real Time qPCR of Pri-miRNAs

Total RNA was reverse transcribed (RT) to cDNA by using the High Capacity cDNA Reverse Transcription Kit (Applied Biosystem, cat #4368814) as previously described. The pri-miRs primers used for qPCR were TaqMan Gene Expression Assays (Applied Biosystem, Life Technologies, Carlsbad Calif., cat #4427012) specific for either: mmu-miR-143 (ID # Mm03306564_pri), rno-miR-143 (ID # Rn03466026_pri), mmu-miR-133b (ID # Mm03307410_pri), rno-miR-133b (ID # Rn03465381_pri) and GAPDH (ID # Rn01775763_g1). GAPDH was used as the endogenous control. Quantification was done using TaqMan Gene Expression Master Mix (Applied Biosystem, cat #4369016).

Real Time qPCR of Mature miRNAs

Total RNA was RT to specific cDNA by using the TaqMan MicroRNA Reverse Transcription Kit (Applied Biosystem, cat #4366596). Stem-loop RT primers and primers used for qPCR were TaqMan microRNA Assays (cat # PN4427975) specific for either: mmu-miR-143-3p (ID #002249), rno-143-3p (ID #000466), hsa-133b-3p (ID #002247) (which recognizes rat and mice), U6 snRNA (ID #001973), U87 (ID #001712) and snoRNA135 (ID #001230). U6, U87 and snoRNA135 were used as endogenous miRNA controls. Quantification was done using TaqMan Universal PCR MasterMix No AmpErase (Applied Biosystem, cat #4324018). The expression of pri-miRs and mature miRNAs was quantified using a CFX96 Touch™ apparatus from BIO-RAD and Multiplate™ Low-Profile 96-Well Unskirted PCR Plates (BIO-RAD, cat # MLL9601). All reactions were run in duplicate. Background controls consisted in replacing the cDNA with water. Forty five cycles of amplification were done. Data (Ct values) from all qRT-PCR experiments were analyzed using a comparative ΔΔCt method.

Transfection of DRG Cultures Used for Electrophysiological, Calcium Imaging and Neurite Outgrowth Measurements

Freshly dissociated primary DRG neuronal cultures (1.5-2 hours following platting) were transfected with either the microRNA or the microRNA-Hairpin inhibitors (anti-miRNA) (33 nM) by using Lipofectamine 2000 (Invitrogen) according to the product instructions. The miR-133b-3p (C-320457-03-0002), miR-133b-3p-hairpin inhibitor (IH-320457-04-0002), miR-143-3p (C-320375-03-0002), miR-143-3p-hairpin inhibitor (IH-320375-05-0002), miR-1a-3p (C-320456-03-0005), and miR-1a-3p-hairpin inhibitor (IH-320456-03-0005) were from Fisher Scientific. Fluorescence Oligo was used to confirm that the cells were transfected. Control cells were exposed to all the manipulations and lipofectamine. The next day following transfection the medium was replaced with NBA medium supplemented with 2% B27, penicillin (100 U/ml), streptomycin (100 μg/ml), 0.5 mM Glutamax, and 5-10% FCS. FCS was pre-depleted of RNA as previously described (Shelke et al., J Extracell Vesicles 2014; 3). Excitability and the KCl-evoked transient increase in [Ca²]_{cyt} were measured at 2-4 days following transfection, and neurite outgrowth was measured 3 days following transfection.

Calcium Imaging

Cells were loaded with the fluorescent Ca²⁺ indicator Fura-2 AM (5 μM)(Molecular Probes, Eugene, Oreg., USA). Coverslips were mounted on an open perfusion chamber (250 μL volume, type RC-21BRFS, or typeRC-25F; Warner Instruments, Hamden, Conn., USA). The chamber was continuously perfused (250 μL/min) with a hepes-buffer (in mM: 140 NaCl, 5 KCl, 5 NaHCO₃, 10 HEPES, 1 MgCl₂, 2 CaCl₂ and 10 glucose, pH7.4). Measurements were done at 35° C., cells were equilibrated for 30 min prior to any stimulation. Cells were stimulated with a 10 second pulse of KCl followed by a 5 second pulse of hepes buffer applied directly on top of the cells through a puffer (puffer pressure: 1 pSi) puffer valve-link 8.2 system, AutomateScientific Inc., San Francisco, Calif., USA). Pulses were electronically controlled (interface: Digidata 1440A, software: Clampfit 10, from Axon Instruments). A sequence of KCl pulses (50 mM, 20 mM, 30 mM, 50 mM) separated by 10 minutes were applied to each coverslip. Only cells that responded to the two 50 mM KCl pulses (the initial one and the last one) were used. Measurements were done by using the ATTO (Atto Instruments, Rockville, Md.) system. The excitation wavelengths for Fura-2 (340 nm and 380 nm, 150 ms exposure each) were applied every 1.25 seconds and generated by passing a transmitted light source through separate filters (340- and 380-nm filters). The emitted fluorescence from individual cells was filtered with a fluorescence barrier filter (475 nm) and collected in a camera (Hamamatsu Orca-ER, C4742-95-12ER, Monochrone 12-bit), with Metafluor software (Molecular Devices). The relative changes in [Ca²⁺]_{cyt} are given by the ratio of the emission of Fura-2 at the barrier filter generated by the a excitation at 340 nm and 380 nm (ratio (340/380)). Experiments were conducted at 32-35° C. The data was analyzed using Metafluor and Clampfit 10. For each miRNA and anti-miRNA we used 3 separate preparations, and for control 6 separate preparations. For each preparation we had 2-3 coverslips per condition (control, miRNA and anti-miRNA). In a given day measurements were done for “control”, “miRNA” and corresponding “anti-miRNA” treated cells.

Electrophysiology Measurements

Electrophysiological recordings were performed under current-clamp by using the whole cell conformation. Intracellular solution (in mM): 140 KCl, 1 MgCl₂, 0.5 CaCl₂, 10 EGTA, 10 Hepes, 4 Mg-ATP, 0.4 Na-GTP, pH 7.2). Extracellular solution (in mM): 150 NaCl, 5 KCl, 2.5 CaCl₂, 1 MgCl₂, 10 Hepes, pH 7.4. Borosilicate pipettes (1.5 mm outer diameter; 0.86 mm inner diameter with filaments, World Precision Instruments, Inc, Sarasota, Fla.) with resistances of 1 to 3 MΩ were used. Under current clamp, 8-18 steps (150-300 ms duration) of current pulses of increasing magnitude (0.05 nA) were applied from the Vr (resting potential). Changes in membrane potential were measured using a 2-electrode voltage clamp amplifier Axopatch 200B (Axon Instruments, Foster City, Calif.; Molecular Devices, LLC, Sunnyvale, Calif.). Output from the current clamp amplifier was sent to a microcomputer using a data acquisition interface (Digidata 1440A; Axon Instruments). Signals were filtered at 2.5 or 5 kHz (Axopatch 200B) and sampled at 10-50 kHz. For analysis the traces were filtered at 1 kHz. Pclamp 10.4 (Axon Instruments) was used for data acquisition and data analyses. Experiments were conducted at room temperature. Number of separate DRG cell culture preparations: Control: 8 preparations (two of them also used for Ca²⁺ imaging); miR-133b-3p: 5 preparations (two of them also used for Ca²⁺ imaging); anti-miR-133b-3p: 6 preparations (two of them also used for Ca²⁺ imaging); miR-143-3p: 8 preparations (three of them also used for Ca²⁺ imaging); anti-miR-143-3p: 5 preparations (two of them also used for Ca²⁺ imaging).

Immunostaining

Coverslips with attached cells were rinsed with warm (37° C.) PBS and fixed with 4% paraformaldehyde for 20 min at room temperature (RT). Cells were rinsed three times with PBS, and permeabilized with 0.3% Triton X-100 in PBS for 15 min at RT. Cells were incubated with 1% bovine serum albumin for 30 min. Then they were incubated overnight (4° C.) with anti-β-III tubulin (rabbit polyclonal ABCam 18207) at 0.3 μg/ml in 2% normal donkey serum (NDS) in PBS. The next day coverslips were incubated with the secondary antibody Donkey anti-Rabbit Alexa-546 (1:1500 dilution in 2% NDS, Molecular Probes) for 1 hour at RT. Cells were counterstained with the nuclear stain Hoechst 33342 (Sigma Aldrich (10 μg/ml in PBS, 15 min RT). After each step cells were rinsed with PBS two times. Negative controls were incubated in the absence of primary antibody. Coverslips were mounted using Aqua Poly/Mount (#18606, Polyscienced Inc, Bayonne, N.J., USA) and stored at 4° C. Images were captured (and analyzed with a Zeiss Axiovert 200 (Germany) inverted microscope equipped with fluorescence and Normaski optics.

### Results

Rats that do not develop chronic pain following a peripheral nerve injury display up-regulation of miR-133b-3p and miR-143-3p

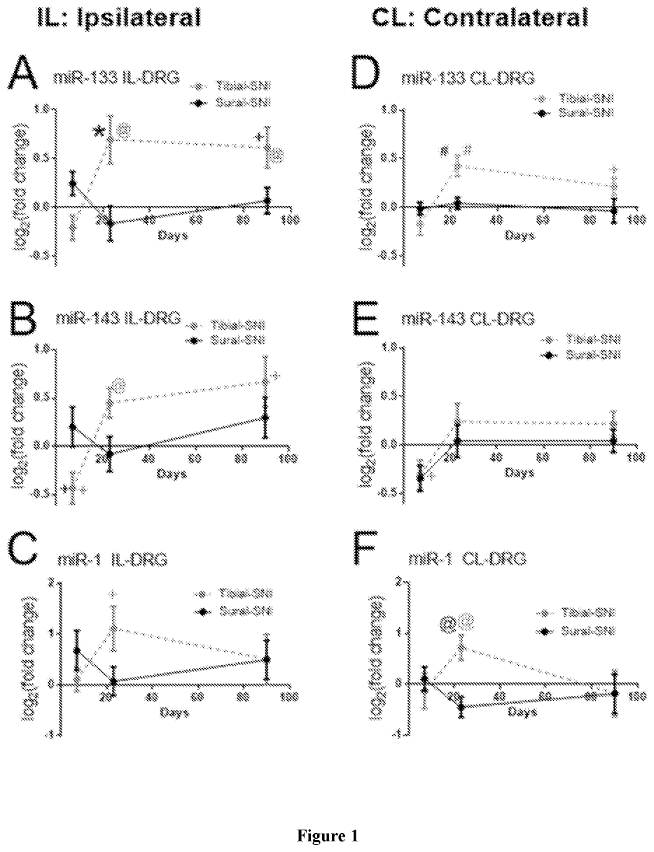

The sural-SNI model has a neuropathic pain phenotype that closely resembles the clinical outcome of surgical patients who develop chronic neuropathic pain. In the sural-SNI, mechanical and cold hypersensitivities develop and are maintained over time, blocking the nerve impulses before and over one week after nerve injury does not prevent the development of chronic neuropathic pain. In contrast, the tibial-SNI model has a neuropathic pain phenotype that more closely resembles the majority of surgical patients who undergo a procedure, that may suffer acute pain but fail to develop chronic neuropathic pain. The allodynia/hypersensitivity that develops in the rat hind paw from transecting some or all of the sciatic nerve branches is evoked by stimulation of the nerve terminals of the spared sciatic nerve branches and from the saphenous nerve that undergoes functional changes as a result of interactions with the injured sciatic nerve fibers. In rats, the sensory neurons of the saphenous nerve are located in the L3-DRG, and those of the sciatic nerve are located in the L4- and L5-DRG. Hence, the level of expression of miR-133b-3p and miR-143-3p was measured over time in L3-L5 DRG derived from sural-SNI and tibial-SNI rats. We found that in the ipsilateral (IL) DRG derived from animals that do not develop chronic neuropathic pain, tibial-SNI rats, the level of expression of these miRNAs was up-regulated at day 23 post injury as compared to those derived from sham animals, and this up-regulation was maintained elevated at least up to day 90 post injury, (FIGS. 1A and B). There was no significant change in the expression of these miRNAs in DRG derived from animals that develop chronic pain, sural-SNI rats as compared to those derived from sham animals (FIGS. 1A and B). Interestingly, in the contralateral (CL) DRGs derived from tibial-SNI, but not those derived from sural-SNI, there was also an up-regulation of these two miRNAs, but to a lower yet still to a significant extent for miR-133b-3p (FIGS. 1C and D). Hence, the sustained up-regulation in the expression of miR-133b-3p and miR-143-3p is distinctly associated with nerve-injured animals that do not develop persistent neuropathic pain.

A Single Intrathecal (i.t.) Injection of miR-143-3p or miR-133b-3p, but not of miR-1a-3p, on the Day of the Injury Prevents the Development of Persistent Mechanical and Cold Allodynia

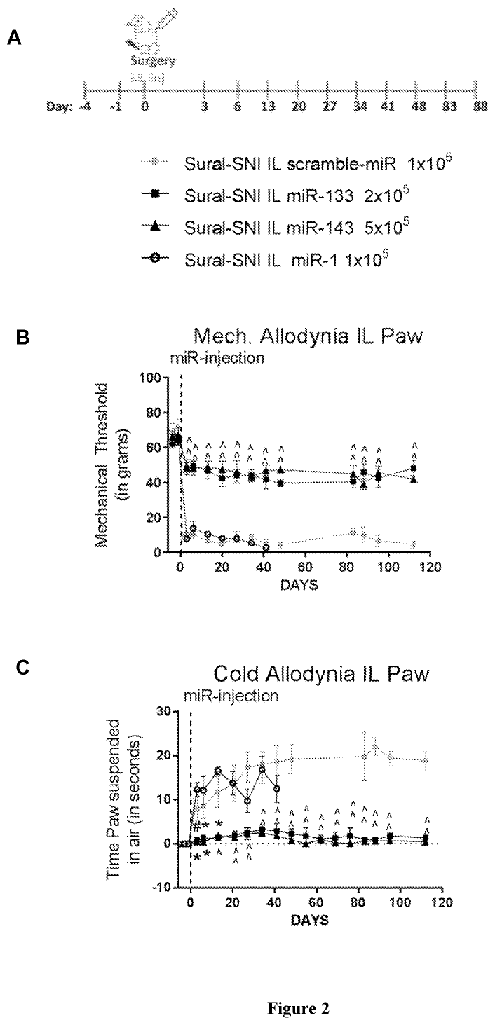

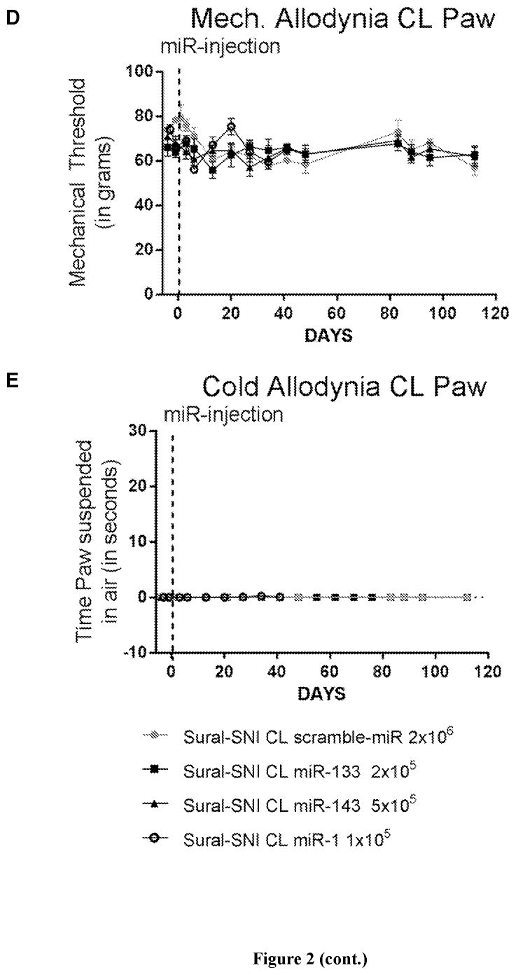

We used measurements of mechanical and cold hypersensitivity/allodynia (increase in the response to an innocuous stimulation) as surrogates for neuropathic pain. The effectiveness of miR-143-3p, miR-133b-3p, and miR-1a-3p in preventing the development of allodynia was tested in the sural-SNI model. The microRNAs were delivered using a single i.t. injection (between L4 and L5) of replication-deficient lentiviral (LV) vector constructs expressing the corresponding pri-miRNA precursor (LV-miR-143-3p, LV-miR-133b-3p, LV-miR-1a-3p). In rats, i.t. injections reach both the spinal cord and the closely located DRG. In separate experiments we confirmed that i.t. injections reached the DRG by using methylene blue (not shown) and by measuring the levels of the miRNA precursor (shown below). Single i.t. injections were done immediately (10-15 minutes) following the surgery (FIG. 2A). Sural-SNI animals, injected with the control/scramble LV-vector (LV-scramble miR) developed mechanical allodynia and cold allodynia in the hind paw ipsilateral (IL) to the injury (FIGS. 2B and C, blue lines) but not in the hind paw CL to the injury (FIGS. 2D and E). The level of mechanical and cold allodynia of the CL paw was not significantly different to that measured prior to the surgery and injection with the LV-scramble miR. Sural-SNI animals injected with a single i.t. injection of either LV-miR-133b-3p or miR-143-3p on the day of the injury never developed strong mechanical or cold allodynia (FIGS. 2B and C). These miRNAs did not affect the sensitivity to mechanical or cold in the CL paws (FIGS. 2D and 2E). Sural-SNI animals injected with a single i.t. injection of LV-miR-1a-3p on the day of the injury did not affect the development of mechanical allodynia and cold allodynia in the hind paw IL to the injury. Hence, a single i.t. injection of either LV-miR-133b-3p or miR-143-3p, but not of miR-1a-3p, on the day of the injury was sufficient to prevent the development of strong acute and strong chronic mechanical and cold allodynia in the IL paw following a peripheral nerve injury, without affecting the sensitivity of the CL paw.

A Single i.t. Injection of miR-143-3p or miR-133b-3p on Day 3 Post-Injury has Different Effects on Mechanical and Cold Allodynia.

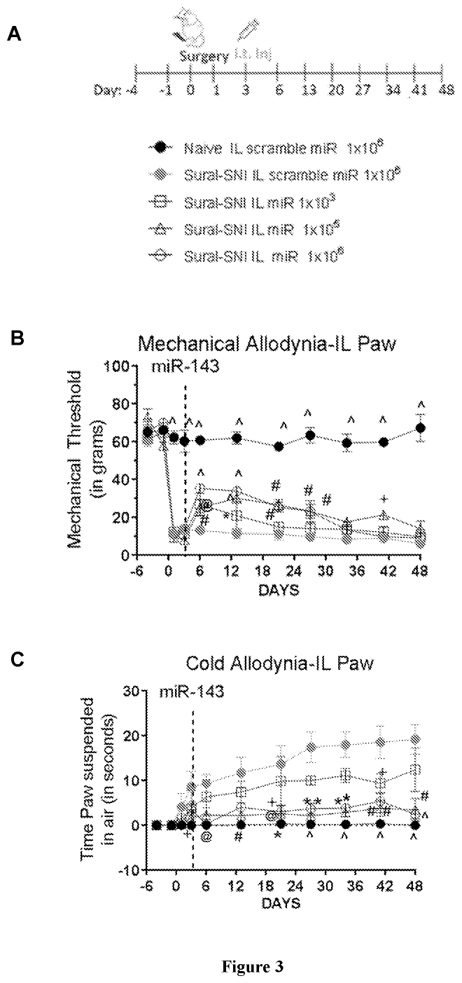

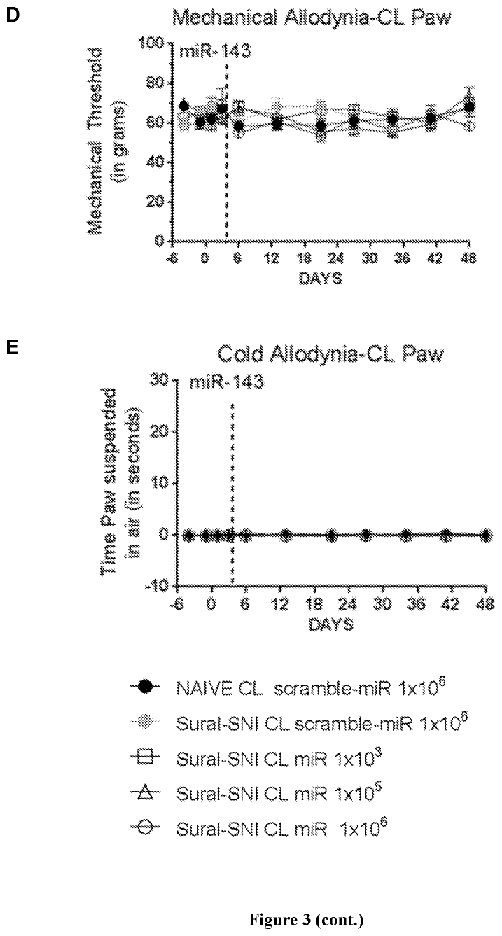

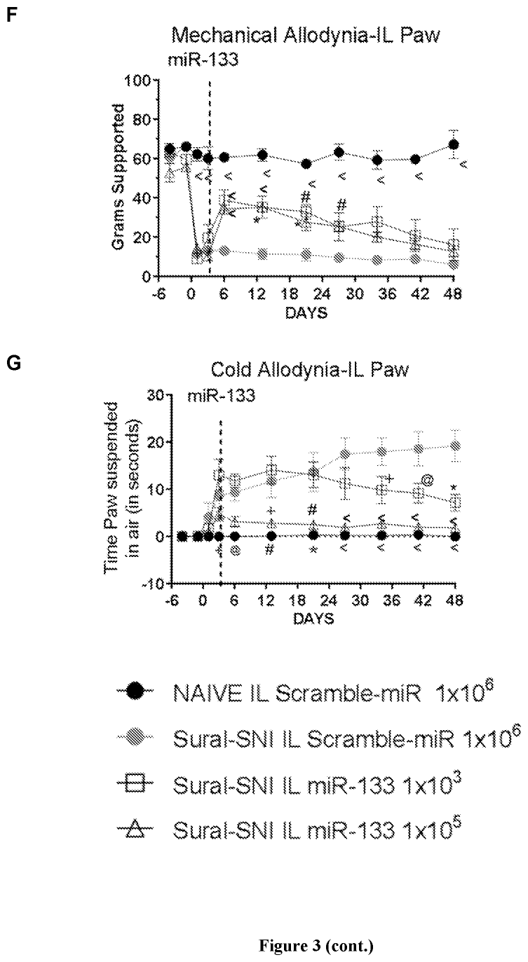

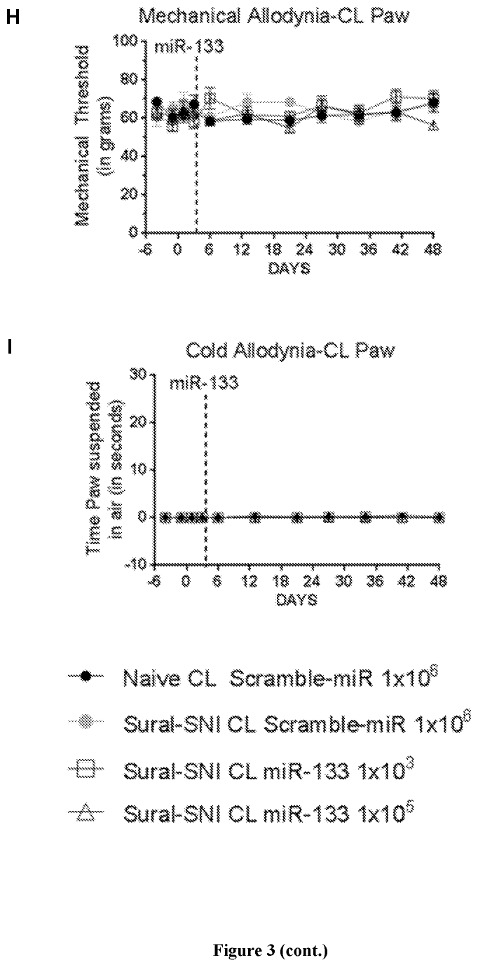

We also investigated whether a single i.t. injection of either LV-miR-133b-3p or miR-143-3p at day 3 post-injury could reverse post-injury allodynia and prevent the development of sustained allodynia (FIG. 3A). In naïve rats the magnitude of mechanical and cold responses was the same before and following the i.t injection of LV-scramble miR (FIGS. 3B and C, black circles) and comparable to that displayed by the contralateral (CL) paws in sural-SNI rats also injected with LV-scramble miRNA (FIGS. 3D and E). We found that a single i.t. injection of the LV-miR-143-3p on day 3 post-injury could reverse the post-injury evoked level of mechanical and cold allodynia in a dose-dependent manner (FIGS. 3B and C). However, there were two major differences: the effect on mechanical allodynia was transient (lasting about 15 days) while the effect on cold allodynia was sustained (maintained through the entire observation period), and the maximal reduction in mechanical allodynia was ˜50%, while that in cold allodynia was more than 90% (FIG. 3B,C). Similar results were observed with LV-miR-133b-3p (FIG. 2F-I). Hence, a single i.t. injection of either LV-miR-133b-3p or miR-143-3p on day 3 post-injury transiently and partially reversed post-injury mechanical allodynia, while it fully reversed post-injury-evoked cold allodynia and still prevented the development of sustained cold allodynia in sural-SNI rats.

A Single i.t. Injection of miR-1a-3p on Day 3 Post-Injury Did not Reverse Mechanical or Cold Allodynia.

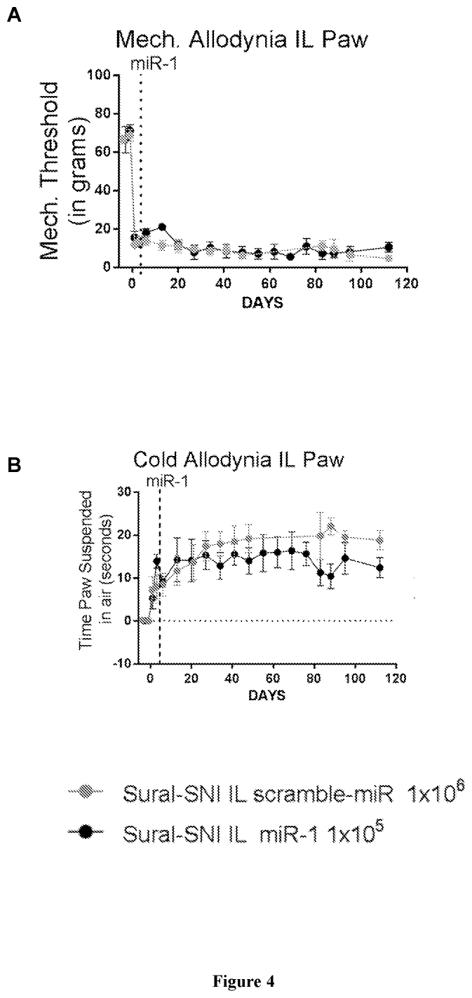

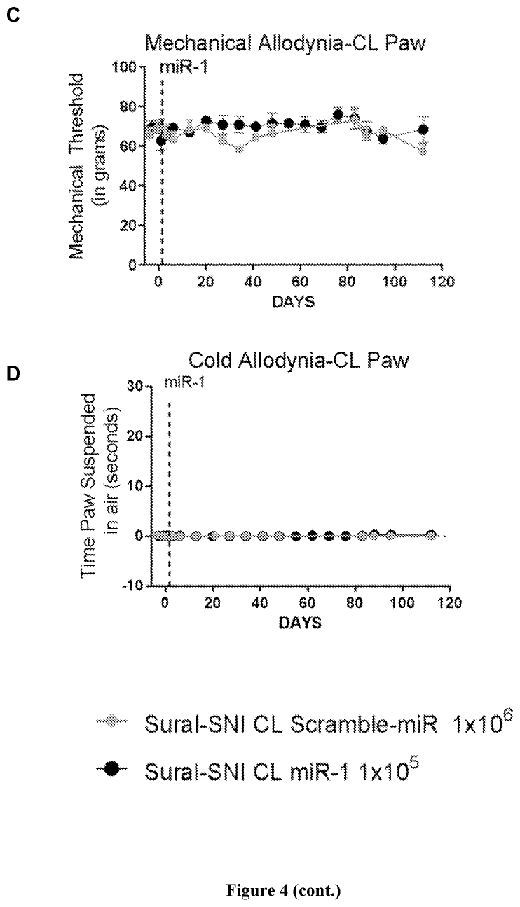

We found that a single i.t. injection of the LV-miR-1a-3p on day 3 post-injury did not reverse the post-injury evoked level of mechanical and cold allodynia in the ipsilateral (IL) paw (FIGS. 4A and B), neither affected the sensation level in the contralateral (CL) paw (FIGS. 4 C and D).

A Single i.t. Injections on Day 3 Post-Injury of miR-133 with miR-1 (miR-133+miR-1) or miR-143 with miR-1 (miR-143+miR-1), but not of miR-133 with miR-143 (miR133+miR-143), Produced a Sustained Recovery of Mechanical and Cold Allodynia.

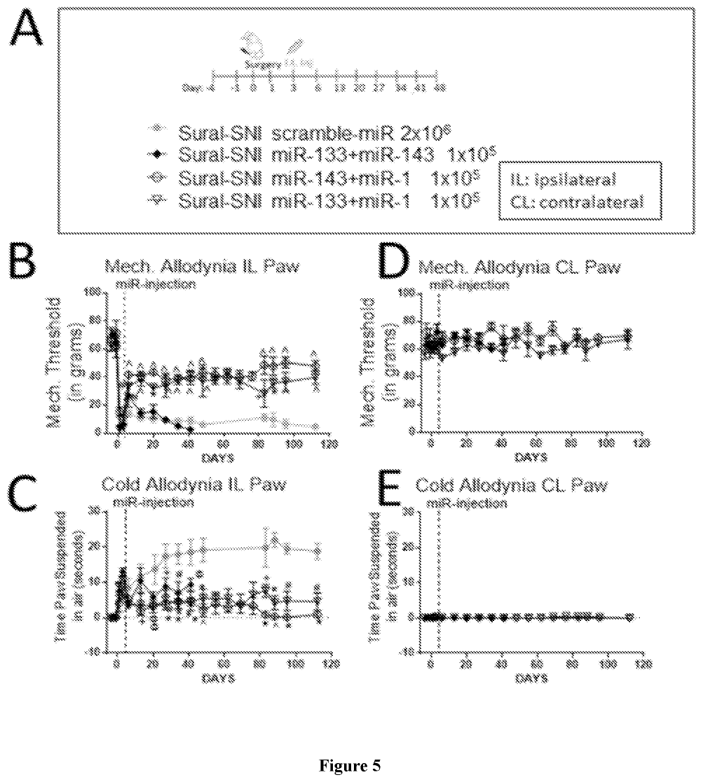

We found that single i.t. injections on day 3 post-injury of miR-133 with miR-1 (miR-133+miR-1) or miR-143 with miR-1 (miR-143+miR-1), but not of miR-133 with miR-143 (miR133+miR-143), produced a sustained recovery of mechanical and cold allodynia in the ipsilateral (IL) paw (FIGS. 5A and B), and did not affect the sensation level in the contralateral (CL) paw (FIGS. 5C and D).

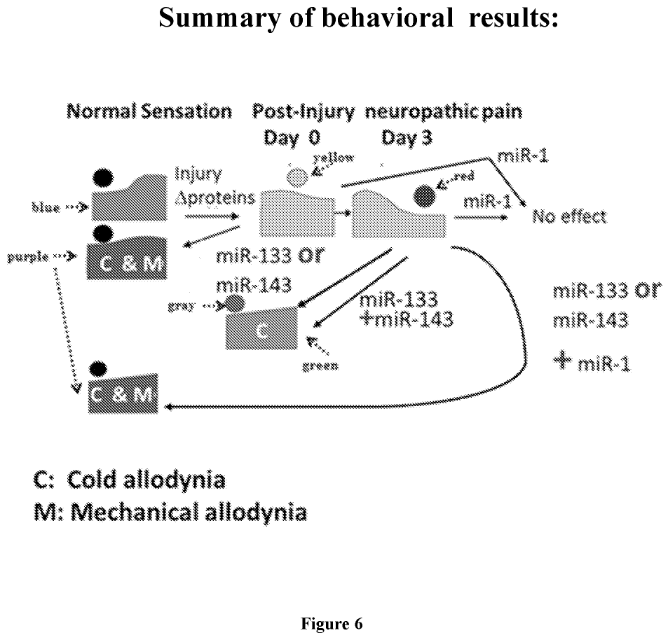

The results indicate that miR-143-3p and miR-133b-3p can sufficiently compensate for the injury-induced molecular changes that result in mechanical and cold hypersensitivity following sural-SNI when applied the day of the injury. At later times following nerve injury (day 3 post-injury) these miR-143 and miR-133 by themselves although they still produced a sustained decrease in cold hypersensitivity they only produced a transient decrease in mechanical hypersensitivity. However, a mixture of miR-143 or miR-133 with miR-1, produced a sustained decrease in mechanical and cold allodynia when applied at later times following nerve injury (day 3 post-injury). In line with the notion that normal sensation is maintained by the equilibrium of the action of many proteins, and that hypersensitivity results from a maladaptation of many of those actions, we show in FIG. 6 a schematic interpretation of our results. The action of many ion channels, transporters, receptors etc, results in an equilibrium state (blue shape) that allows for normal sensation (black circle). Injury induces changes in the expression of multiple proteins, leading to maladaptive equilibrium states (orange shapes), that result in hypersensitivity/neuropathic pain (yellow circle: strong mechanical allodynia and mild cold allodynia; red circle: strong mechanical and strong cold allodynia). Early treatment with either miR-133b-3p or miR-143-3p, (but not with miR-1a-3p), reverses many of the injury-evoked changes allowing for sufficient reversal of the maladaptive equilibrium (purple shapes) and thus allowing sustained recovery/maintenance of normal sensation (black circles). Later treatments with either miR-133b-3p, miR-143-3p, or miR-133b-3p with miR-143-3p, appear to sufficiently reverse in a sustained manner the maladaptive equilibrium (green shape) for some (cold allodynia) but not all (mechanical allodynia) sensations (grey circle). However, later treatment with either miR-133b-3p or miR-143-3p in combination with miR-1a-3p, can still reverse many of the injury-evoked changes allowing for sufficient reversal of the late maladaptive equilibrium.

A Single i.t. Injection of LV-miRs does not Lead to a Recovery of Toe-Spread in the Ipsilateral Paw

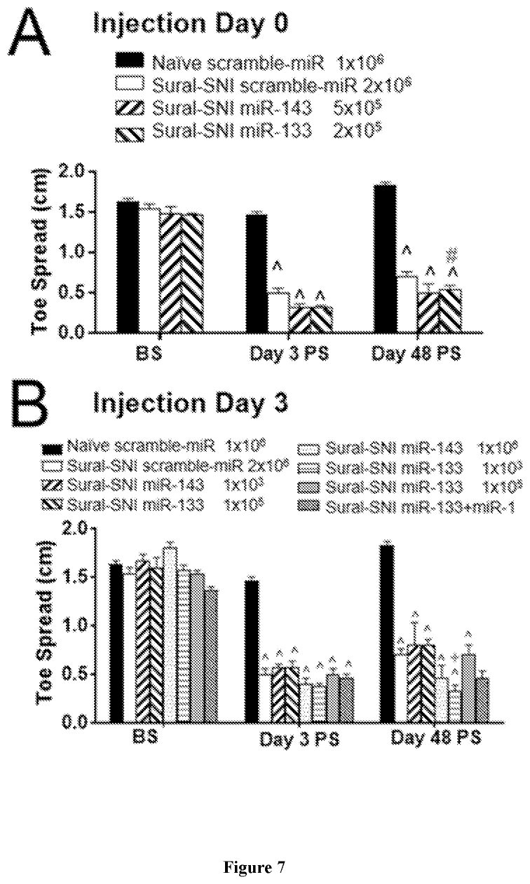

We found that administering a single i.t. injection of LV-miR-143-3p or LV-miR-133b-3p, either on the day of the surgery (FIG. 7A) or on day 3 post-surgery (FIG. 7B), did not significantly affect the decrease in toe-spread observed in sural SNI rats. The same was found when administering a single i.t. injection of LV-miR-143-3p with miR-1a-3p, LV-miR-133b-3p with LV-miR-1a-3p, or LV-miR-143-3p with LV-miR-133b-3p on day 3 post-surgery (data has been collected, need to make the graph).

Expression of LV-miRs

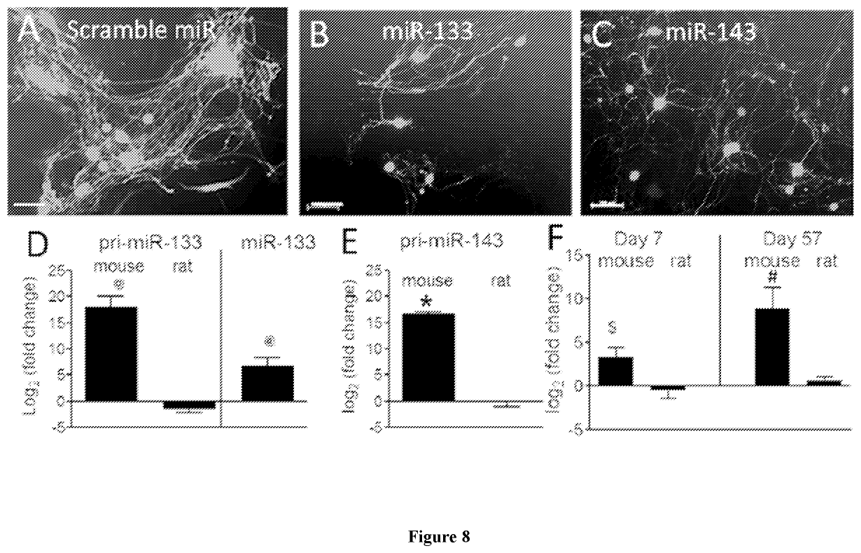

The LV-vector constructs contained a GFP marker gene. For each viral preparation their infectivity (TU/ml; TU: Transduction units) was determined by using HEK cells and counting the number of GFP positive cells (not shown), and their capacity to infect primary sensory neurons was determined by using adult rat DRG cultures (GFP positive cells FIG. 8A-C). The mature form of miR-133b-3p (UUUGGUCCCCUUCAACCAGCUA) (SEQ ID NO:1) is the same in rat and mice, and that of miR-143-3p is one residue longer in rat than in mice (mmu-UGAGAUGAAGCACUGUAGCUC (SEQ ID NO:4); rno-UGAGAUGAAGCACUGUAGCUCA) (SEQ ID NO:3). The LV vectors contain murine miRNA precursors, thus the level of expression of the rat-pri-miR (endogenous) and mouse-pri-miR (LV-vector) was measured to show that the LV-vectors were expressed and processed in rat DRG. The levels of mouse-pri-miR-133b-3p and mature miR-133b-3p were increased in DRG cultures transfected with LV-miR-133b-3p (FIG. 8D); similarly the levels of mouse-pri-miR-143-3p increased in DRG cultures transfected with LV-miR-143-3p (FIG. 8E). As expected the level of rat-pri-miRs was not affected in DRG cultures transfected with the LV-miRs (FIG. 8D-E). In DRG isolated from sural-SNI rats at day 7 and day 57 following the i.t. injection with LV-miR-133b-p, the levels of mouse-pri-miR-133b-3p were higher than the levels of the endogenous rat-pri-miR-133b-3p (FIG. 8F). This data indicates that the LV-vectors used in this study are expressed and processed in adult rat DRG, and that the expression is maintained over time.

MiR-143-3p and miR-133b-3p Decrease Excitability

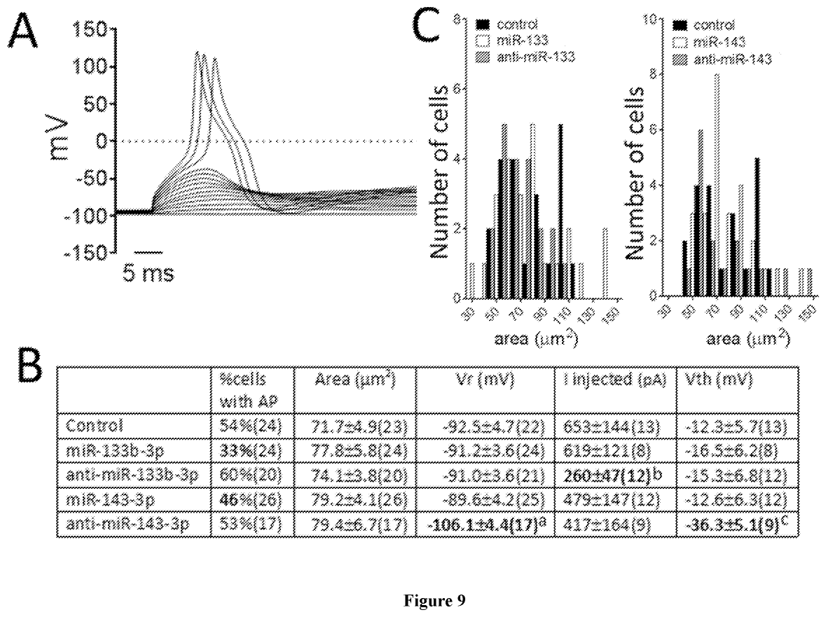

Increases in neuronal excitability have been found to contribute to the initiation and maintenance of chronic neuropathic pain after peripheral nerve injury. We investigated whether changes in the expression of miR-133b-3p and miR-143-3p could affect neuronal excitability of cultured DRG neurons. Under the whole cell current clamp mode we applied current pulses of increasing magnitude from the resting potential to measure the neuron's capacity to evoke an action potential (AP) when transfected with either miRNAs or anti-miRNAs (FIG. 9A). Based on size, the population of neurons assessed across treatment groups was similar (FIG. 9B, C). The percentage of neurons in which an AP could be evoked was lower in DRG cultures treated with the miRNA than when treated with their corresponding anti-miRNA (FIG. 9B). Neurons treated with anti-miR-143-3p showed the most negative resting potential (Vr) and the most negative threshold potential (Vth, minimum depolarization needed to generate an AP) (FIG. 9B). Such a negative resting potential suggests that transporters may be involved (e.g. H+ transporter) in defining the resting potential on these neurons (as it has been shown for mitochondria, or even some plant cells). Hence, inducing a down-regulation of miR-143 in DRG neurons enhances their excitability, since the neurons required a smaller depolarization (Vth) to generate an AP. Cells treated with anti-miR-133b-3p required a lower magnitude of current injected (I injected) to generate an AP (FIG. 6B), which also indicates an enhancement in neuronal excitability. Therefore, increasing the expression of miR-133b-3p or miR-143-3p with the i.t. injection of the corresponding LV-miR may contribute to the observed decrease/prevention in neuropathic pain by decreasing/preventing the injury-evoked increase in excitability observed in the sural-SNI model.

MiR-143-3p and miR-133b-3p Enhance the Neuronal Depolarization-Evoked Transient Increase in [Ca²⁺]_{cyt}

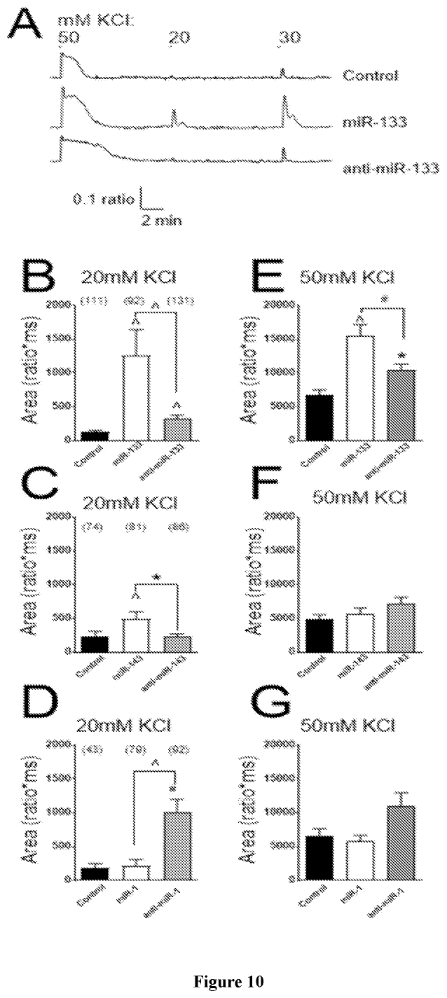

Peripheral nerve injury is known to decrease Ca²⁺ stores in the endoplasmic reticulum, and decrease mitochondrial Ca²⁺ content and Ca²⁺ buffering capacity. We investigated whether miR-133b-3p and miR-143-3p could affect the regulation of intracellular Ca²⁺ stores by measuring their effects on depolarization-evoked transient increases in [Ca²⁺]_{cyt} in cultured DRG neurons. Fura-2 was used as the cytoplasmic Ca²⁺ indicator and depolarization was evoked with KCl. The KCl-evoked transient increase in [Ca²⁺]_{cyt} is initiated by the opening of voltage-dependent Ca²⁺ channels, and its shape is defined by a complex system of buffers, pumps, and release mechanisms from various intracellular Ca²⁺ stores. In primary sensory neurons the magnitude of the contribution of distinct intracellular Ca²⁺ stores depends on the depolarization level; during exposure to 20-30 mM KCl or to a short electrical stimulation, the endoplasmic reticulum is the major contributor to the resulting transient increase in [Ca²⁺]_{cyt}. Following exposure to higher KCl (50 mM) or to a long electrical stimulation, mitochondria also become a large contributor to the resulting transient increase in [Ca²⁺]_{cyt}, initially by sequestering Ca²⁺ and subsequently by a slow release of Ca²⁺ which produces a shoulder in the [Ca²⁺]_{cyt} transient and prolongs the return to resting [Ca²⁺]_{cyt} levels. We examined the effect of miR-133b-3p and miR-143-3p following depolarization with 20, 30 and 50 mM KCl. The 50 mM KCl pulse produced a large and long-lasting transient increase in [Ca²⁺]_{cyt}, and the 20 mM and 30 mM KCl pulses produced much smaller and shorter responses (FIG. 10A). The area of the transient increase in [Ca²⁺]_{cyt} was taken as a measure of the overall increase in [Ca²⁺]_{cyt} evoked by the KCl stimulation. Treatment with miR-133b-3p resulted in an increase in the area following stimulation with 20 mM, 30 mM, or 50 mM KCl (FIG. 10B). Treatment with miR-143-3p also resulted in an increase in the area at 20 mM and 30 mM, but not at 50 mM KCl (FIG. 10C). These results indicate that in primary sensory neurons miR-133b-3p and miR-143-3p increase the endoplasmic reticulum contribution to the depolarization evoked increase in [Ca²⁺]_{cyt}, which appears to be stronger for miR-133b-3p than for miR-143-3p. That miR-133b-3p also increased the magnitude of the 50 mM KCl response suggests that miR-133b-3p may also increase the mitochondrial contribution to the depolarization-evoked increase in [Ca²⁺]_{cyt}. The latter may involve either a direct action on increasing the mitochondrial buffering capacity or an indirect action as a result of the miR-133b-3p-mediated larger increase in Ca²⁺ release from the endoplasmic reticulum. Therefore, increasing the expression of miR-133b-3p or miR-143-3p with the i.t. injection of the corresponding LV-miR may contribute to the observed decrease/prevention in neuropathic pain by decreasing/preventing the injury-evoked decrease in intracellular Ca²⁺ stores.

MiR-133b-3p has a Positive Effect while miR-143-3p has a Negative Effect on Neurite Outgrowth

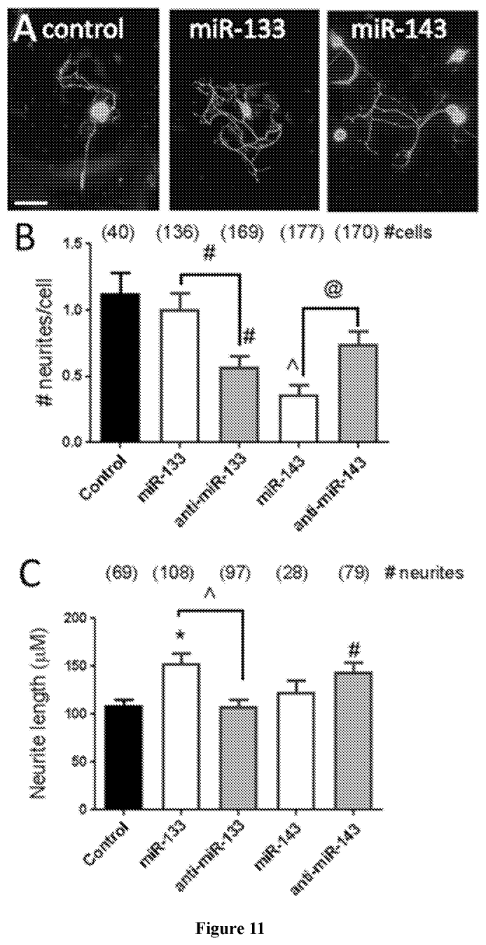

We investigated whether miR-133b-3p and miR-143-3p could affect regeneration of primary sensory neurons by measuring their effect on neurite outgrowth. Treatment with miR-133b-3p did not affect the number of neurites per cell (FIG. 11A,B) but increased the neurite length (FIG. 11C) compared to control cells and to cells treated with anti-miR-133b-3p. In contrast, treatment with miR-143-3p decreased the number of neurites per cell (FIG. 11A,B) but had no effect in the neurite length (FIG. 11C) compared to control cells and to cells treated with anti-miR-143-3p following a peripheral nerve injury.

The Actions of miR-133b-3p and miR-143-3p on 3′UTR-Scn2b and 3′UTR-Trpm8 are Similar and Differ from Those of miR-1a-3p.

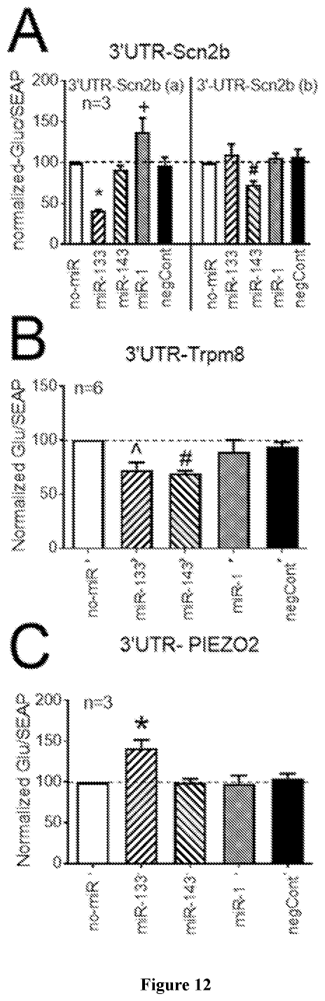

The actions of miR-133b-3p, miR-143-3p and miR-1a-3p most likely result from their actions on multiple proteins. Based on the behavioral studies we postulate that the molecular actions of miR-133b-3p and miR-143-3p may be similar and complementary to those of miR-1a-3p. To test this we examined the actions of these miRs on several 3′UTR (untranslated regions) regions of mRNA that encode proteins that have been previously associated with neuropathic pain. MiR 3′UTR target clones containing a Gaussian luciferase reporter gene were used to investigate whether these miRs targeted the 3′UTR regions of mRNAs that encode for the sodium voltage-gated channel beta subunit 2 (Scn2b) a protein that promotes neuronal excitability, the transient receptor potential cation channel subfamily M member 8 (TRPM8) a transducer of cold somatosensation, or the Piezo2 channel a transducer of mechanosensation. Since the 3′UTR-Scn2b region is very long, two clones were used the 3′UTR-Scn2b(a) and the Scn2b(b). With the 3′UTR-Scn2b(a), miR-133b-3p reduced the expression of the reporter gene, while miR-1a-3p enhanced the expression of the reporter gene (FIG. 12A, left panel). With the 3′UTR-Scn2b(b), miR-143-3p reduced the expression of the reporter gene (FIG. 12A, right panel). Hence, with the 3′UTR-Scn2b, miR-133 and miR-143 reduced the expression of the reporter gene, by interacting on different regions, while miR-1 enhanced the expression of the reporter gene. With the 3′UTR-TRPM8, miR-133 and miR-143 reduced the expression of the reporter gene, and miR-1 had no effect (FIG. 12B). When using the 3′UTR-Piezo2, miR-133 increased the expression of the reporter gene, while miR-143 and miR-1 had no effect on the expression of the reporter gene (FIG. 12C).

Summary of Results

We found a sustained upregulation of miR-133b-3p and miR-143-3p in nerve-injured animals that do not develop chronic pain. By mimicking this up-regulation via intrathecal injections of LV-miR vectors in a nerve injury model where animals do develop chronic pain, we effectively prevented the development of persistent cold and mechanical allodynia. When injected individually, the magnitude and duration of the miRNAs' effects depended on when the injections were done relative to the nerve injury; if the i.t. injections (miR-143 or miR-133) were done on the day of the injury, they prevented the development of strong acute and strong chronic mechanical and cold allodynia in the ipsilateral paw over the entire observation period. If the animals were injected at 3 days post-injury with miR-143 or miR-133, a transient and partial reversal of post-injury mechanical allodynia was observed, but post-injury-evoked cold allodynia was significantly decreased for the entire observation period. The difference in effect on mechanical allodynia suggests that following a peripheral nerve injury, there is an early critical period during which the application of either of these two miRs can prevent the development of sustained allodynia. In the sural-SNI model this critical period appears to be shorter for mechanical allodynia (<1 day) than for cold-allodynia (>3 days). In contrast, injection with miR-1 had no effect whether injected on the day of the injury or 3 days post-injury.

When injected in combination, either miR-133 with miR-1, or miR-143 with miR-1, at 3 days post-injury, these mixture of miRs produced a sustained decrease in both mechanical and cold allodynia. In contrast, injection of miR-133 with miR-143 still did not reverse mechanical allodynia.

In cultured sensory neurons, increasing the level of either miR-143-3p or miR-133b-3p decreased the probability of evoking an action potential (AP). However, these miRs appear to affect neuronal excitability through different actions. Neurons treated with anti-miR-143-3p showed the most negative resting potential (Vr) and threshold potential (Vth). Hence under this condition a smaller depolarization (Vth) is required to generate an AP, indicating that a down-regulation of miR-143-3p enhances neuronal excitability. Such an increase in excitability could in part result from the also observed more negative Vr which by itself will lead to an increase in the proportion of functionally available (in the resting closed-state) voltage-dependent Na⁺ channels. Actions in the expression of these or other channels may also be involved. On the other hand, neurons treated with anti-miR-133b-3p did not show a change in the Vr, however they required a lower magnitude of current injected to generate an AP, which also indicates an enhancement in neuronal excitability. These results suggest that a down-regulation of both of these miRs may contribute to the observed increase in primary sensory neuron excitability in the sural-SNI model. Moreover, it also suggests that the upregulation of both of these miRs as displayed by L3-L5-DRG derived from tibial-SNI rats (FIG. 1A,B), may prevent the development of chronic allodynia in tibial-SNI rats in part by limiting/preventing injury-induced increase in neuronal excitability. Decreasing neuronal excitability via treatment with miR-133 and 143 could contribute to the observed decrease/prevention in neuropathic pain in the sural-SNI model.

We also found that treatment with either miR-133b-3p or miR-143-3p enhanced the magnitude of the transient increase in [Ca²⁺]_{cyt} following stimulation with 20 mM, and 30 mM KCl. In primary sensory neurons the endoplasmic reticulum is the major contributor of the resulting transient increase in [Ca²⁺]_{cyt} when evoked by 20-30 mM KCl. Hence miR-133b-3p and miR-143-3p appear to enhance the Ca²⁺ content of the endoplasmic reticulum. Treatment with miR-133b-3p, but not miR-143-3p also increased the magnitude of the [Ca²⁺]_{cyt} transient increase evoked by 50 mM KCl. miR-133b-3p may also enhance the mitochondrial Ca²⁺ contribution. The latter may involve either a direct action on increasing the mitochondrial Ca²⁺ buffering capacity or an indirect action as a result of the miR-133b-3p-mediated apparent larger increase in Ca²⁺ release from the endoplasmic reticulum (larger responses to 20 mM and 30 mM KCl). Our results suggest that treatment with miR-133b-3p or miR-143-3p enhances intracellular Ca²⁺ stores in primary sensory neurons and this effect correlates with the observed decrease/prevention in neuropathic pain in the sural-SNI model following i.t. injection with the LV-miRs.

With respect to neurite outgrowth, treatment with miR-133b-3p had a positive effect by promoting an increase in the neurite length while treatment with miR-143-p had a negative effect by decreasing the number of neurites per neuron. Since both miRs decreased mechanical and cold allodynia, their differential effect on neurite outgrowth may not contribute to the evoked-hypersensitivity we measured in sural-SNI.

The various actions of miR-133 and miR-143 appear to be sufficient to produce a persistent decrease in cold allodynia whether these miRNAs were applied on the day of the injury or 3 days post-injury. However, such actions only appear to be sufficient to produce a persistent decrease in mechanical allodynia if injections are done on the day of the injury. However, the combined actions of miR-133 with miR-1, or of miR-143 with miR-1 appear to be sufficient to produce a persistence decrease of mechanical allodynia (in addition to cold allodynia) when applied at 3 days post-injury. The various actions of miR-1, do not appear to be sufficient to produce a persistent decrease in either mechanical or cold allodynia when applied at either the day of the injury or 3 days post-injury.

Toe-spread changes in rats after peripheral nerve injury mainly assess motor function of the sciatic nerve. We found that the decrease in toe-spread was maintained even when animals displayed complete recovery from allodynia following miRNA treatment; hence these miRNAs appear to be specific to sensory, and not motor, recovery. It is possible that the beneficial motor effect of miR-133b-3p involves an action on injured-spinal cord cells. However, it is also possible that a beneficial motor effect was not detected in the sural-SNI model since in this model about two mm of nerve segment are removed, potentially limiting the miRNA action on motor neurons.

The i.t. application delivers the LV-miR vector to L-DRGs on both sides of the spinal cord and to the spinal cord itself. However the presence of the LV-miRs in the contralateral-DRG and spinal cord, do not appear to affect the sensitivity of the contralateral paws.

Based on the behavioral studies the molecular actions of miR-133b-3p and miR-143-3p appear to be sufficient to prevent the development of sustained neuropathic pain if injected on the day of the injury. However, when they are injected at later times following the injury, even in combination (miR-133b-3p+miR-143-3p), their molecular actions only promote sustained recovery of some neuropathic pain manifestations. The molecular actions of miR-1a by themselves are not sufficient to treat neuropathic pain; but they appear to complement those of miR-133b-3p and of miR-143-3p to allow for the sustained recovery of neuropathic pain when co-injected at later times following nerve injury. Since the combination of miR-133b-3p and miR-143-3p result in the same behavioral changes as when injected individually on day 3 post-injury, it appears at least some of the “pain relevant” molecular actions of miR-133b-3p and miR-143-3p may be similar and complementary to those of miR-1a-3p. This was examined by measuring the miR actions on the depolarization-evoked transient increase in cytoplasmic calcium ([Ca²⁺]_{cyt}) and on three 3′UTR regions of mRNAs that encode for proteins previously associated with neuropathic pain.

In summary, miR-143-3p or miR-133b-3p alone or in conbinaiton with miR-1a-3p represent new ‘recovery’ rather than palliative pharmacological agents that can be administered at different times after peripheral nerve injury, and have long durations of efficacy to prevent or significantly attenuate neuropathic pain. This is an important finding since the development of chronic neuropathic pain after surgery is associated with peripheral nerve injury, and effective treatment of chronic neuropathic pain remains relatively unsuccessful in many surgical patients.

While the present invention has been described through various specific embodiments, routine modification to these embodiments will be apparent to those skilled in the art, which modifications are intended to be included within the scope of this disclosure.

1. A method of treating neuropathic pain comprising administering to an individual in need of treatment an effective amount of one or more of the following polynucleotides:
i) miR-133b-3p miRNA or precursor thereof, or a DNA polynucleotide encoding the miRNA or the precursor thereof, or an expression vector encoding the miR-133b-3p miRNA or the precursor thereof; and
ii) miR-143-3p miRNA or precursor thereof, or a DNA polynucleotide encoding the miRNA or the precursor thereof, an expression vector encoding the miR-143-3p miRNA or the precursor thereof.

2. The method of claim 1, further comprising administering to the individual, miR-1a-3p miRNA or a precursor thereof, or a DNA polynucleotide encoding the miR-1a-3p miRNA or the precursor thereof, or an expression vector encoding miR-1a-3p miRNA or the precursor thereof.

3. The method of claim 1, wherein the method comprises administering miR-133b-3p miRNA and miR-143-3p miRNA in separate compositions.

4. The method of claim 2, wherein the method comprises administering miR-133b-3p miRNA, miR-143-3p miRNA, and miR-1a-3p miRNA in separate compositions.

5. The method of claim 1, wherein the polynucleotides are administered via intrathecal delivery.

6. The method of claim 1, wherein the neuropathic pain is allodynia.

7. The method of claim 6, wherein the allodynia is caused by nerve injury and the composition comprising:
i) miR-133b-3p miRNA or precursor thereof, or a DNA polynucleotide encoding the miRNA or the precursor thereof, or an expression vector encoding the miR-133b-3p miRNA or the precursor thereof; and/or
ii) miR-143-3p miRNA or precursor thereof, or a DNA polynucleotide encoding the miRNA or the precursor thereof, an expression vector encoding the miR-143-3p miRNA or the precursor thereof,
wherein i) and/or ii) are administered within 3 days of the injury.

8. The method of claim 7, wherein i) and/or ii) are administered within 3 days, 2 days, 1 day, 12 hours, 6 hours, or 2 hours of the injury

9. The method of claim 6, wherein the allodynia is caused by nerve injury and a composition comprising:
i) miR-133b-3p miRNA or precursor thereof, or a DNA polynucleotide encoding the miRNA or the precursor thereof, or an expression vector encoding the miR-133b-3p miRNA or the precursor thereof; and
ii) miR-1a-3p miRNA or a precursor thereof, or a DNA polynucleotide encoding the miR-1a-3p miRNA or the precursor thereof, or an expression vector encoding miR-1a-3p miRNA or the precursor thereof,
wherein i) and ii) are administered within 7 days of injury.

10. The method of claim 6, wherein the allodynia is caused by nerve injury and the composition comprises:
i) miR-143-3p miRNA or precursor thereof, or a DNA polynucleotide encoding the miRNA or the precursor thereof, an expression vector encoding the miR-143-3p miRNA or the precursor thereof; and
ii) miR-1a-3p miRNA or a precursor thereof, or a DNA polynucleotide encoding the miR-1a-3p miRNA or the precursor thereof, or an expression vector encoding miR-1a-3p miRNA or the precursor thereof,
wherein i) and ii) are administered within 7 days of injury.

11. The method of claim 6, wherein the allodynia is caused by nerve injury and the composition comprises:
i) miR-143-3p miRNA or precursor thereof, or a DNA polynucleotide encoding the miRNA or the precursor thereof, an expression vector encoding the miR-143-3p miRNA or the precursor thereof;
ii) miR-133b-3p miRNA or precursor thereof, or a DNA polynucleotide encoding the miRNA or the precursor thereof, or an expression vector encoding the miR-133b-3p miRNA or the precursor thereof; and
iii) miR-1a-3p miRNA or a precursor thereof, or a DNA polynucleotide encoding the miR-1a-3p miRNA or the precursor thereof, or an expression vector encoding miR-1a-3p miRNA or the precursor thereof,
wherein i) ii) and iii) are administered within 7 days of injury.

12. The method of claim 9, wherein i), ii), and iii) are administered after 3 days of injury.

13. The method of claim 6, wherein the allodynia is mechanical allodynia.

14. The method of claim 6, wherein the allodynia is cold allodynia.

15. An expression vector encoding an miRNA or a precursor thereof, wherein the miRNA is miR-133b-3p, miR-143-3p, or miR-1a-3p.

16. The expression vector of claim 15, wherein the expression vector is a lentiviral vector or herpes simplex virus (HSV) vector.

17. A pharmaceutical composition comprising the expression vector of claim 15.


SEQUENCE LISTING (22 sequences)

SEQ ID NO: 1 - RNA, 22 nt, Homo sapiens
uuugguccccuucaaccagcua

SEQ ID NO: 2 - RNA, 22 nt, Homo sapiens
gcuggucaaacggaaccaaguc

SEQ ID NO: 3 - RNA, 22 nt, Rattus norvegicus
ugagaugaagcacuguagcuca

SEQ ID NO: 4 - RNA, 21 nt, Homo sapiens
ugagaugaagcacuguagcuc

SEQ ID NO: 5 - RNA, 21 nt, Mus musculus
ggugcagugcugcaucucugg

SEQ ID NO: 6 - RNA, 22 nt, Homo sapiens
ggugcagugcugcaucucuggu

SEQ ID NO: 7 - RNA, 22 nt, Rattus norvegicus
uggaauguaaagaaguguguau

SEQ ID NO: 8 - RNA, 22 nt, Homo sapiens
uggaauguaaagaaguauguau

SEQ ID NO: 9 - RNA, 22 nt, Rattus norvegicus
gcacauacuucuuuauguaccc

SEQ ID NO: 10 - RNA, 23 nt, Mus musculus
acauacuucuuuauaugcccaua

SEQ ID NO: 11 - RNA, 23 nt, Mus musculus
acauacuucuuuauauacccaua

SEQ ID NO: 12 - RNA, 25 nt, Homo sapiens
guacauacuucuuuauguacccaua

SEQ ID NO: 13 - RNA, 119 nt, Mus musculus
ccuccaaagggaguggcccccugcucuggcuggucaaacggaaccaaguccgucuuccugagagguuugguccccuucaaccagcuacagcagggcuggcaaagcucaauauuuggaga

SEQ ID NO: 14 - RNA, 84 nt, Rattus norvegicus
cccugcucuggcuggucaaacggaaccaaguccgucuuccugagagguuugguccccuucaaccagcuacagcagggcuggcaa

SEQ ID NO: 15 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 16 - RNA, 105 nt, Rattus norvegicus
gcggagcgccugucucccagccugaggugcagugcugcaucucuggucaguugggagucugagaugaagcacuguagcucaggaagggagaagauguucugcagc

SEQ ID NO: 17 - RNA, 63 nt, Mus musculus
ccugaggugcagugcugcaucucuggucaguugggagucugagaugaagcacuguagcucagg

SEQ ID NO: 18 - RNA, 106 nt, Homo sapiens
gcgcagcgcccugucucccagccugaggugcagugcugcaucucuggucaguugggagucugagaugaagcacuguagcucaggaagagagaaguuguucugcagc

SEQ ID NO: 19 - RNA, 87 nt, Rattus norvegicus
ugccuacucagagcacauacuucuuuauguacccauaugaacauagaaugcuauggaauguaaagaaguguguauuuuggguaggua

SEQ ID NO: 20 - RNA, 72 nt, Mus musculus
ucagagcacauacuucuuuauguacccauaugaacauucagugcuauggaauguaaagaaguauguauuuug

SEQ ID NO: 21 - RNA, 71 nt, Homo sapiens
ugggaaacauacuucuuuauaugcccauauggaccugcuaagcuauggaauguaaagaaguauguaucuca

SEQ ID NO: 22 - RNA, 85 nt, Homo sapiens
accuacucagaguacauacuucuuuauguacccauaugaacauacaaugcuauggaauguaaagaaguauguauuuuugguaggc